(19) United States
(12) Patent Application Publication
(10) Pub. No.: US 2014/0058271 A1
(43) Pub. Date: Feb. 27, 2014

(54) SYSTEMS AND METHODS FOR ANALYSIS AND TREATMENT OF A BODY LUMEN

(71) Applicant: Angiolight, Inc., Medford, MA (US)

(72) Inventors: S. Eric Ryan, Hopkinton, MA (US); Jing Tang, Arlington, MA (US); Richard Gambale, Tyngsboro, MA (US)

(73) Assignee: Angiolight, Inc., Medford, MA (US)

(21) Appl. No.: 14/060,042

(22) Filed: Oct. 22, 2013

Related U.S. Application Data
(63) Continuation of application No. 12/784,482, filed on May 20, 2010, now abandoned.
Application No. 12/784,482 is a continuation-in-part of application No. 11/537,258, filed on Sep. 29, 2006.
(60) Provisional application No. 61/180,068, filed on May 20, 2009.
(60) Provisional application No. 61/310,337, filed on Mar. 4, 2010.
(60) Provisional application No. 60/824,915, filed on Sep. 8, 2006.
(60) Provisional application No. 60/823,812, filed on Aug. 29, 2006.
(60) Provisional application No. 60/821,623, filed on Aug. 7, 2006.
(60) Provisional application No. 60/761,649, filed on Jan. 24, 2006.
(60) Provisional application No. 60/722,753, filed on Sep. 30, 2005.

(51) Int. Cl. A61B 5/00

(52) U.S. Cl. CPC A61B 5/0086; A61B 5/0075; A61B 5/0071; A61B 5/0066; A61B 5/6853
USPC 600/477

(57) ABSTRACT

A system for analyzing a body lumen including a flexible conduit that is elongated along a longitudinal axis, the flexible conduit having a proximal end and a distal end; at least one delivery waveguide and at least one collection waveguide extending along the flexible conduit, a transmission output of the at least one delivery waveguide and a transmission input of the at least one collection waveguide located along a distal portion of the conduit; a spectrometer connected to the at least one delivery waveguide and the at least one collection waveguide, the spectrometer configured to perform diffuse reflectance spectroscopy, wherein the spectrometer emits at least one primary radiation signal of a wavelength having an absorption coefficient of between about 8 cm⁻¹ and about 10 cm⁻¹ when transmitted through a highly aqueous media; a controller system configured to calculate at least one of an extent, area, and volume of highly aqueous media based on the amount of absorption of the at least one primary radiation signal measured through the highly aqueous media by the spectrometer.

### RELATED APPLICATIONS

This application is a continuation of U.S. patent application Ser. No. 12/784,482, filed May 20, 2010, which is a continuation-in-part of U.S. patent application Ser. No. 11/537,258, filed Sep. 29, 2006, which claims the benefit of U.S. Provisional Application No. 60/824,915, filed Sep. 8, 2006, U.S. Provisional Application No. 60/823,812, filed Aug. 29, 2006, U.S. Provisional Application No. 60/821,623, filed Aug. 7, 2006, U.S. Provisional Application No. 60/761,649, filed Jan. 24, 2006, and U.S. Provisional Application No. 60/722,753, filed Sep. 30, 2005, the entire contents of each being herein incorporated by reference in their entirety. This application further claims the benefit of U.S. Provisional Application No. 61/180,068, filed May 20, 2009 and U.S. Provisional Application No. 61/310,337, filed Mar. 4, 2010, the entire contents of each being herein incorporated by reference in their entirety. This application is related to U.S. patent application Ser. No. 11/834,096, filed on Aug. 6, 2007, published as U.S. Patent Application Publication No. 2007/0270717 A1, U.S. Provisional Application No. 61/019,626, filed Jan. 8, 2008, U.S. Provisional Application No. 61/025,514, filed Feb. 1, 2008, U.S. Provisional Application No. 61/082,721 filed Jul. 22, 2008, U.S. patent application Ser. No. 12/350,870, filed Jan. 8, 2009, published as U.S. Patent Application Publication No. 2009/0187108 A1, U.S. patent application Ser. No. 12/561,756, filed Sep. 17, 2009, the contents of each being incorporated herein by reference in their entirety. This application is further related to PCT Application No. PCT/US10,35677, filed on even date herewith, titled “SYSTEMS AND METHODS FOR ANALYSIS AND TREATMENT OF A BODY LUMEN”, by S. Eric Ryan, et al., Attorney Docket No. COR-22CPPCTA, and PCT Application No. PCT/US10,35682, filed on even date herewith, titled “SYSTEMS AND METHODS FOR ANALYSIS AND TREATMENT OF A BODY LUMEN”, by S. Eric Ryan, et al., Attorney Docket No. COR-22CPPCTB, the entire contents of each being herein incorporated by reference in their entirety.

### BACKGROUND OF THE INVENTION

1. Field of the Invention

The present inventive concepts relate generally to systems and methods for the analysis and treatment of a lumen. More particularly, the present inventive concepts relate to balloon catheter systems that are used to perform methods of analysis and angioplasty of endovascular lesions.

2. Description of the Related Art

With the continual expansion of minimally-invasive procedures in medicine, one procedure that has been highlighted in recent years has been percutaneous transluminal angioplasty, or “PTA”. The most prevalent use of this procedure is in the coronary arteries, which is more specifically called a percutaneous coronary transluminal angioplasty, or “PTCA”. These procedures utilize a flexible catheter with an inflation lumen to expand, under relatively high pressure, a balloon at the distal end of the catheter to expand a stenotic lesion.

The PTA and PTCA procedures are now commonly used in conjunction with expandable tubular structures known as stents, and an angioplasty balloon is often used to expand and permanently place the stent within the lumen. An angioplasty balloon utilized with a stent is referred to as a stent delivery system. Conventional stents have been shown to be more effective than angioplasty alone in maintaining patency in most types of lesions and also reducing other near-term endovascular events. A risk with a conventional stent, however, is the reduction in efficacy of the stent due to the growth of the tissues surrounding the stent which can again result in the stenosis of the lumen, often referred to as restenosis. In recent years, new stents that are coated with pharmaceutical agents, often in combination with a polymer, have been introduced and shown to significantly reduce the rate of restenosis. These coated stents are generally referred to as drug-eluting stents, though some coated stents have a passive coating instead of an active pharmaceutical agent.

With the advent of these advanced technologies for PTA and PTCA, there has been a substantial amount of clinical and pathology literature published about the pathophysiologic or morphologic factors within an endovascular lesion that contribute to its restenosis or other acute events such as thrombosis. These features include, but are not limited to, collagen content, lipid content, calcium content, inflammatory factors, and the relative positioning of these features within the plaque. Several studies have been provided showing the promise of identifying the above factors through the use of visible and/or near infrared spectroscopy, i.e., across wavelengths ranging between about 250 to 2500 nm, including those studies referenced in U.S. Publication No. US2004/0111016A1 by Casscells, III et al., U.S. Publication No. US2004/0077950A1 by Marshik-Geurts et al., U.S. Pat. No. 5,304,173 by Kittrell et al., and U.S. Pat. No. 6,095,982 by Richards-Kortum, et al., the contents of each of which are herein incorporated by reference. However, there are very few, if any, highly safe and commercially viable applications making use of this spectroscopic data for combining diagnosis and treatment in a PTA or PTCA procedure.

In addition, dynamic and optimal control over the expansion of the balloon during angioplasty procedures is very limited, including during pre-dilation of the vasculature prior to stent delivery, dilation during stent delivery, and post-dilation after delivery of a stent. For example, under-expansion of an angioplasty balloon may require deployment of an additional catheter and stent in order to complete the desired treatment and/or to ensure that an under-expanded stent is not blocking blood flow through a vessel, which can complicate procedures, resulting in increased risks, and added expense. Information about the apposition and expansion of the balloon against the vessel walls during these procedures could therefore be highly useful for mitigating these risks.

Typical technologies used for monitoring angioplasty and stenting procedures include angiography by fluoroscopy, which supplies an X-ray image of the blood flow within a lumen. However, this technology has a limited resolution of about 300 micrometers. As a result, many angioplasty and stenting procedures over-expand the lumen, which can result in unnecessary trauma and damage to the lumen wall, complicating post-deployment recovery, and increasing the likelihood of re-closure of the lumen (restenosis).

Angioscope technology is also generally used for identifying a stenosis, but provides no information about the endovascular wall of the plaque. Some important diseases located on non- or minor stenosis regions, such as a vulnerable plaque which is fatal to a patient life, are often missed. Moreover, radiation delivered by an angiography procedure can have negative side-effects on patients.

Other technologies, such as intravascular ultrasound, require expensive additional catheters and potentially dangerous additional procedures that can cause more harm than good and still not supply sufficient information about the plaque to be beneficial. Currently, there are needs for physicians to gain this useful information about the lumen wall, including accurately locating diseased tissue for purposes of conducting angioplasty procedures in an accurate, cost-effective, and efficient manner that presents a reasonable risk profile for the patient.

Conventional balloon catheters are not generally used for purposes other than for performing traditional angioplasty procedures including pre-dilation of the vasculature prior to stent delivery, stent delivery, and post-stent delivery dilation. A capability that is not presently available in conventional balloon catheters, which would be highly valuable before, during, and after such procedures, would be the ability to assess the optimal type of stent and/or stent coating, if any, to be deployed within a patient. The availability of the aforementioned pathophysiologic or morphologic factors could be used to help such assessments.

Furthermore, the level and uniformity of expansion of balloons during such procedures is only roughly determined, e.g., with use of an angiogram and a balloon expansion estimation charts, and is often unnecessarily exceeded in order to avoid issues associated with under-expansion as previously discussed. Over-expansion, however, carries its own risks including, for example, rupture of a lesion or excessive damage to a weakened vessel wall. For these reasons, stent deployment may be avoided altogether and substituted with less risky but less effective procedures.

Prior use of optical fibers within an angioplasty catheter permit functions such as visualization to occur, but limited information from such techniques can be obtained. Conventional balloon catheters generally have no capacity to collect any information beyond the surface of the endovascular wall, which can be critical to proper diagnosis and treatment of diseased vessels. While lower-pressure balloon catheters are available to occlude the blood flow proximal to the optical analysis window of a catheter, no lumen expansion is performed and no analysis can be performed within the balloon itself. Other systems support the use of optical feedback within a balloon catheter to atraumatically minimize the blood path between the balloon catheter and the endovascular wall. However, these systems likewise provide no ability to perform a complete optical analysis of the lumen wall.

### SUMMARY OF THE INVENTION

Embodiments of the present inventive concepts are directed to systems and methods that provide physicians performing lumen-expansion procedures with useful information about the lumen wall without any significant increase in their procedure time or cost, and with little to no additional risk to the patient. Included are a number of implementations of distal fiber-optic configurations to optimally facilitate analysis of the lumen wall and angioplasty balloon characteristics. These implementations also provide manufacturability and relatively low-cost production required for a disposable medical device.

In an embodiment, the distal fiber optical configuration distributes at least one delivery waveguide and at least one collection waveguide with distal ends arranged such that, upon expansion of the balloon catheter in a body lumen, the distal waveguide ends can be positioned proximate to the perimeter of the catheter's treatment end by one or more expandable, flexible whisker arms. The embodiment permits positioning of the waveguide ends with little or no media fluid or bodily fluid positioned between the distal waveguide ends and the lumen wall.

In an embodiment, the apparatus includes a single balloon to which the waveguide ends are held against by the whiskers such that fiber ends remain proximate to the balloon's wall during expansion with fluid media.

In an embodiment, the delivery and collection ends of fibers of the optical configuration are adapted for near-field, wide scope use. The adaptation is particularly advantageous where the delivery and/or collection ends are to be positioned closely to targeted tissue and/or blood during deployment as in various embodiments described herein. In an embodiment, at least one delivery and/or a collection end is manufactured using a controlled etching process. In an embodiment, fiber tips are formed through emersion in a liquefied etchant such as, for example, hydrofluoric acid over a pre-determined period of time.

In one embodiment, optical analysis of the plaque is performed within the same catheter utilized for angioplasty during a PTA or PTCA procedure. This optical analysis could include, but not limited to, Raman spectroscopy, infrared spectroscopy, fluorescence spectroscopy, optical coherence reflectometery, optical coherence tomography, but most preferably, diffuse-reflective, near-infrared spectroscopy. The embodiment provides optical analysis, and thus the pathophysiologic or morphologic features diagnosis, of a plaque during an angioplasty procedure without any significant additional cost, risk, or work for the physician. With access to this information, a physician could potentially choose from a selection of drug-eluting stents with different doses or agents, or even select a stent without a drug if indicated. During typical angioplasty procedures performed on a patient, including pre-dilation of a lumen, stent delivery, and/or post-dilation, a physician could learn more about the general status of the patient's vasculature which can guide systemic therapies. New emerging technologies such as bioabsorbable stents could be enabled by the embodiments of the invention to optimize their use in the correct type of lesion.

In addition to obtaining information useful to diagnosis, an embodiment obtains information about the level of expansion of the balloon within the lumen. In an embodiment, information is collected about the amount of blood between the balloon wall and a lumen or between a delivery output and collection input of waveguides so as to determine if and when the balloon is fully apposed to the lumen wall and/or to help diagnose and locate pathophysiologic or morphologic factors including the size of the lumen. Information about the balloon with respect to the lumen can be used to control the balloon's expansion so that it does not under-expand or over-expand during treatment or for selecting an appropriately sized stent for subsequent placement. In certain circumstances, a lesion and/or deposit can cause an angioplasty balloon to become mal-apposed upon expansion. In an embodiment, levels of blood are measured about the balloon perimeter to help diagnose hard lesions.

In one aspect, a system for analyzing a body lumen comprises a flexible conduit that is elongated along a longitudinal axis, the flexible conduit having a proximal end and a distal end; at least one delivery waveguide and at least one collection waveguide extending along the flexible conduit, a transmission output of the at least one delivery waveguide and a transmission input of the at least one collection waveguide located along a distal portion of the conduit; a spectrometer connected to the at least one delivery waveguide and the at least one collection waveguide, the spectrometer configured to perform diffuse reflectance spectroscopy, wherein the spectrometer emits at least one primary radiation signal of a wavelength having an absorption coefficient of between about 8 cm⁻¹ and about 10 cm⁻¹ when transmitted through a highly aqueous media; a controller system configured to calculate at least one of an extent, area, and volume of highly aqueous media based on the amount of absorption of the at least one primary radiation signal measured through the highly aqueous media by the spectrometer.

In an embodiment, the at least one primary radiation signal comprises a wavelength between about 1350 nanometers and about 1850 nanometers.

In an embodiment, the at least one primary radiation signal further comprises a wavelength of about 1550 nanometers.

In an embodiment, the spectrometer is further configured to perform spectroscopy of at least one reference radiation signal of a wavelength having an absorption coefficient of less than about 8 cm⁻¹, and wherein the controller system is further configured to calculate a ratio of absorption between the amount of absorption of the at least one primary radiation signal and an amount of absorption of the at least one reference radiation signal measured through the highly aqueous media by the spectrometer in order to calculate the volume of highly aqueous media.

In an embodiment, the at least one reference radiation signal comprises a wavelength having an absorption coefficient of about 1 cm⁻¹ when transmitted through a highly aqueous media.

In an embodiment, the at least one primary radiation signal comprises a wavelength of about 1550 nanometers and the at least one reference radiation signal comprises a wavelength of about 1310 nanometers.

In an embodiment, the system further comprises an angioplasty balloon disposed about a distal portion of the conduit.

In an embodiment, the transmission output of the at least one delivery waveguide and the transmission input of the at least one collection waveguide is located within the angioplasty balloon.

In an embodiment, the transmission output of the at the at least one delivery waveguide and the transmission input of the at least one collection waveguide are translatable along the longitudinal axis of the conduit.

In an embodiment, the transmission output of the at the at least one delivery waveguide and the transmission input of the at least one collection waveguide are radially translatable with respect to the conduit.

In another aspect, a method for treating or analyzing a body lumen comprises: inserting into a body lumen a catheter, the catheter comprising a flexible conduit that is elongated along a longitudinal axis, the flexible conduit having a proximal end and a distal end, at least one delivery waveguide and at least one collection waveguide extending along the flexible conduit, a transmission output of the at least one delivery waveguide and a transmission input of the at least one collection waveguide located along a distal portion of the conduit; maneuvering the conduit into a designated region of the body lumen designated for treatment or analysis; performing spectroscopy, wherein performing spectroscopy comprises: transmitting at least one primary radiation signal through the at least one transmission output, wherein the wavelength of the at least one primary radiation signal has an absorption coefficient of between about 8 cm⁻¹ and 10 cm⁻¹ when transmitted through a highly aqueous media; and collecting the at least one primary radiation signal at the at least one collection waveguide; and measuring at least one of an extent, area, and volume of highly aqueous media about the at least one transmission output and the at least one transmission input with data obtained from the spectroscopy.

In an embodiment, the at least one primary radiation signal comprises a wavelength between about 1350 nanometers and about 1850 nanometers.

In an embodiment, the at least one primary radiation signal further comprises a wavelength of about 1550 nanometers.

In an embodiment, wherein performing spectroscopy further comprises: transmitting at least one reference radiation signal through the at least one transmission output, wherein the wavelength of the at least one reference radiation signal has an absorption coefficient of less then about 8 cm⁻¹ when transmitted through a highly aqueous media; and calculating a ratio of absorption between the amount of absorption of the at least one primary radiation signal and an amount of absorption of the at least one reference radiation signal measured through the highly aqueous media in order to calculate the volume of highly aqueous media.

In an embodiment, the at least one reference radiation signal comprises a wavelength having an absorption coefficient of about 1 cm⁻¹ when transmitted through a highly aqueous media.

In an embodiment, the at least one primary radiation signal comprises a wavelength of about 1550 nanometers and the at least one reference radiation signal comprises a wavelength of about 1310 nanometers.

In an embodiment, the highly aqueous media comprises a saline solution.

In an embodiment, the highly aqueous media comprises blood.

In an embodiment, measuring the volume of highly aqueous media further comprises measuring the volume of expansion of an angioplasty catheter.

In an embodiment, measuring the volume of highly aqueous media further comprises measuring the width of the body lumen.

In an embodiment, during the performance of spectroscopy, at least one of the at least one transmission output and transmission input is positioned contiguously against the conduit.

In an embodiment, during the performance of spectroscopy, at least one of the at least one transmission output and transmission input is positioned adjacent to the body lumen.

Other advantages and novel features, including optical methods and designs of illuminating and collecting an optical signal of a lumen wall through a lumen-expanding balloon, are described within the detailed description of the various embodiments of the present specification.

### BRIEF DESCRIPTION OF THE DRAWINGS

The foregoing and other objects, features, and advantages of the present inventive concepts will be apparent from the more particular description of preferred embodiments, as illustrated in the accompanying drawings in which like reference characters refer to the same elements throughout the different views. The drawings are not necessarily to scale, emphasis instead being placed upon illustrating the principles of the present embodiments.

FIG. 1A is an illustrative view of a catheter instrument for analyzing and medically treating a lumen, in accordance with embodiments of the present inventive concepts.

FIG. 1B is a block diagram illustrating an instrument deployed for analyzing and medically treating the lumen of a patient, in accordance with embodiments of the present inventive concepts.

FIGS. 2A-2F are cross-sectional views illustrating sequential steps of performing a balloon angioplasty procedure, in accordance with embodiments of the present inventive concepts.

FIG. 3A is an illustrative schematic view of a fiber tip being formed in an etchant solution in a method, in accordance with embodiments of the present inventive concepts.

FIG. 3B is an illustrative view of the fiber tip of FIG. 3A, while placed in an etchant solution, in accordance with embodiments of the present inventive concepts.

FIG. 3C is an illustrative schematic view of the fiber tip of FIG. 3A after extraction from an etchant solution, in accordance with embodiments of the present inventive concepts.

FIG. 3D is an illustrative schematic view of a of a recessed fiber tip being placed in a sealant solution, in accordance with embodiments of the present inventive concepts.

FIG. 3E is an illustrative schematic view of the fiber tip of FIG. 3D after extraction from the sealant solution of FIG. 3D, in accordance with embodiments of the present inventive concepts.

FIG. 3F is an illustrative schematic view of the fiber tip of FIG. 3E with sample signal trace lines, in accordance with embodiments of the present inventive concepts.

FIG. 3G is an illustrative view of a reflective coating being applied to the fiber tip of FIG. 3F, in accordance with embodiments of the present inventive concepts.

FIG. 3H is an illustrative view of the fiber tip of FIGS. 3F and 3G with sample signal trace lines after application of a reflective coating, in accordance with embodiments of the present inventive concepts.

FIG. 3I is an illustrative schematic view of a side-fire type of fiber optic tip, in accordance with embodiments of the present inventive concepts.

FIG. 3J is an illustrative view of a reflective coating being applied to the fiber tip of FIG. 3I, in accordance with embodiments of the present inventive concepts.

FIG. 3K is an illustrative view of the fiber tip of FIGS. 3I and 3J with sample signal trace lines after application of a reflective coating, in accordance with embodiments of the present inventive concepts.

FIG. 3L is an illustrative view of a fiber tip with an etched recess, in accordance with embodiments of the present inventive concepts.

FIG. 3M is an illustrative view of the fiber tip of FIG. 3L having a light diffusing covering, in accordance with embodiments of the present inventive concepts.

FIG. 3N is an illustrative view of the fiber tip of FIG. 3L with a light diffusing tip, in accordance with embodiments of the present inventive concepts.

FIG. 4A is an expanded illustrative view of the treatment end of a catheter instrument, in accordance with embodiments of the present inventive concepts.

FIG. 4B is an expanded illustrative view of the treatment end of a catheter instrument, in accordance with embodiments of the present inventive concepts.

FIG. 5 is an expanded illustrative view of the treatment end of a catheter instrument, in accordance with embodiments of the present inventive concepts.

FIG. 6A is an expanded illustrative view of the treatment end of a catheter instrument, in accordance with embodiments of the present inventive concepts.

FIG. 6B is a cross-sectional view of the catheter of FIG. 6A, taken along section lines I-I′ of FIG. 6A, in accordance with embodiments of the present inventive concepts.

FIG. 7 is an expanded illustrative cross-sectional view of the treatment end of a catheter instrument, in accordance with embodiments of the present inventive concepts.

FIG. 8A is an illustrative schematic of a catheter configuration including two delivery fibers and two collection fibers positioned along the inside surface of a balloon, in accordance with embodiments of the present inventive concepts.

FIG. 8B is an illustrative cross-sectional schematic of the delivery fibers and collection fibers positioned for analyzing the expansion profile of the balloons of FIG. 8A within a lumen, in accordance with embodiments of the present inventive concepts.

FIG. 9A is an expanded illustrative view of the treatment end of a catheter instrument, in accordance with embodiments of the present inventive concepts.

FIG. 9B is a cross-sectional view of the catheter of FIG. 9A, taken along section lines I-I′ of FIG. 9A, in accordance with embodiments of the present inventive concepts.

FIG. 9C is a cross-sectional view of a fiber alignment ring, in accordance with embodiments of the present inventive concepts.

FIG. 10A is an expanded illustrative view of the treatment end of a catheter instrument, in accordance with embodiments of the present inventive concepts.

FIG. 10B is an expanded illustrative view of a fiber alignment fixture, in accordance with embodiments of the present inventive concepts.

FIG. 10C is a cross-sectional view of the fiber alignment fixture of FIG. 10B, taken along section lines I-I′ of FIG. 10B, in accordance with embodiments of the present inventive concepts.

FIG. 10D is an expanded illustrative view of a fiber alignment fixture, in accordance with embodiments of the present inventive concepts.

FIG. 10E is a cross-sectional view of the fiber alignment fixture FIG. 10D, taken along section lines II-II′ of FIG. 10D, in accordance with embodiments of the present inventive concepts.

FIG. 10F is an expanded illustrative view of a fiber alignment fixture, in accordance with embodiments of the present inventive concepts.

FIG. 10G is a cross-sectional view of the fiber alignment fixture FIG. 10F, taken along section lines III-III′ of FIG. 10F, in accordance with embodiments of the present inventive concepts.

FIG. 10H is an expanded illustrative view of a fiber probe arrangement, in accordance with embodiments of the present inventive concepts.

FIG. 11A is an illustrative schematic of an optical source and detector configuration of a catheter, in accordance with embodiments of the present inventive concepts.

FIG. 11B is an illustrative schematic of an optical source and detector configuration, in accordance with embodiments of the present inventive concepts.

FIG. 12A is a logarithmic chart of measured absorption coefficients in water relative to selected wavelengths of light, in accordance with embodiments of the present inventive concepts.

FIG. 12B is a chart comparing the absorption coefficient with the predicted % amount of signal delivered through 4 mm of water, in accordance with embodiments of the present inventive concepts.

FIG. 12C is a chart comparing the predicted change in intensity of light over each 100 mm of travel through water in comparison to the light's absorption coefficient, in accordance with embodiments of the present inventive concepts.

FIG. 13A is an illustrative schematic of a console configuration, in accordance with embodiments of the present inventive concepts.

FIG. 13B is a chart of signals delivered and detected over a period of cycles through the system of FIG. 13A, in accordance with embodiments of the present inventive concepts.

FIG. 13C is a flow chart of pre-programming and operation of a catheter system, in accordance with embodiments of the present inventive concepts.

FIG. 14A is an illustrative view of the distal end of a catheter instrument for manipulating slidable fibers with flexible whiskers, in accordance with embodiments of the present inventive concepts

FIG. 14B is an illustrative view of the distal end of the catheter instrument of FIG. 14A showing the flexible whiskers deployed, in accordance with embodiments of the present inventive concepts.

FIG. 14C is an illustrative view of the distal end of the catheter instrument of FIG. 14A showing the flexible whiskers retracted, in accordance with embodiments of the present inventive concepts.

FIG. 14D is an illustrative view of the distal end of a catheter instrument for manipulating slidable fibers with flexible whiskers, in accordance with embodiments of the present inventive concepts.

FIG. 15A is an illustrative view of the distal end of a catheter instrument with slidable fibers, in accordance with embodiments of the present inventive concepts.

FIG. 15B is a cross-sectional illustrative view of the catheter instrument of FIG. 15A, in accordance with embodiments of the present inventive concepts.

FIG. 16A is an illustrative view of the proximate end of a catheter instrument for manipulating slidable fibers, in accordance with embodiments of the present inventive concepts.

FIG. 16B is a cross-sectional illustrative view of the catheter instrument of FIG. 16A, in accordance with embodiments of the present inventive concepts.

FIG. 16C is a cross-sectional illustrative view of the catheter instrument of FIGS. 16A and 16B, taken along section lines I-I′ of FIG. 16B, in accordance with embodiments of the present inventive concepts.

### DETAILED DESCRIPTION OF EMBODIMENTS OF THE INVENTION

The accompanying drawings are described below, in which example embodiments in accordance with the present inventive concepts are shown. Specific structural and functional details disclosed herein are merely representative. The inventive concepts described herein may be embodied in many alternate forms and should not be construed as limited to example embodiments set forth herein. Accordingly, specific embodiments are shown by way of example in the drawings. It should be understood, however, that there is no intent to limit the present inventive concepts to the particular forms disclosed herein, but on the contrary, the present inventive concepts are to cover all modifications, equivalents, and alternatives falling within the spirit and scope of the claims. Like numbers refer to like elements throughout the description of the figures.

It will be understood that, although the terms first, second, etc. may be used herein to describe various elements, these elements should not be limited by these terms. These terms are used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

It will be understood that when an element is referred to as being “on,” “connected to” or “coupled to” another element, it can be directly on, connected to or coupled to the other element or intervening elements may be present. In contrast, when an element is referred to as being “directly on,” “directly connected to” or “directly coupled to” another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments and is not intended to be limiting of the invention. As used herein, the singular forms “a,” “an” and “the” are intended to include the plural fauns as well, unless the context clearly indicates otherwise. It will be further understood that the teems “comprise,” “comprises,” “comprising,” “include,” “includes” and/or “including,” when used herein, specify the presence of stated features, integers, steps, operations, elements, and/or components, but do not preclude the presence or addition of one or more other features, integers, steps, operations, elements, components, and/or groups thereof.

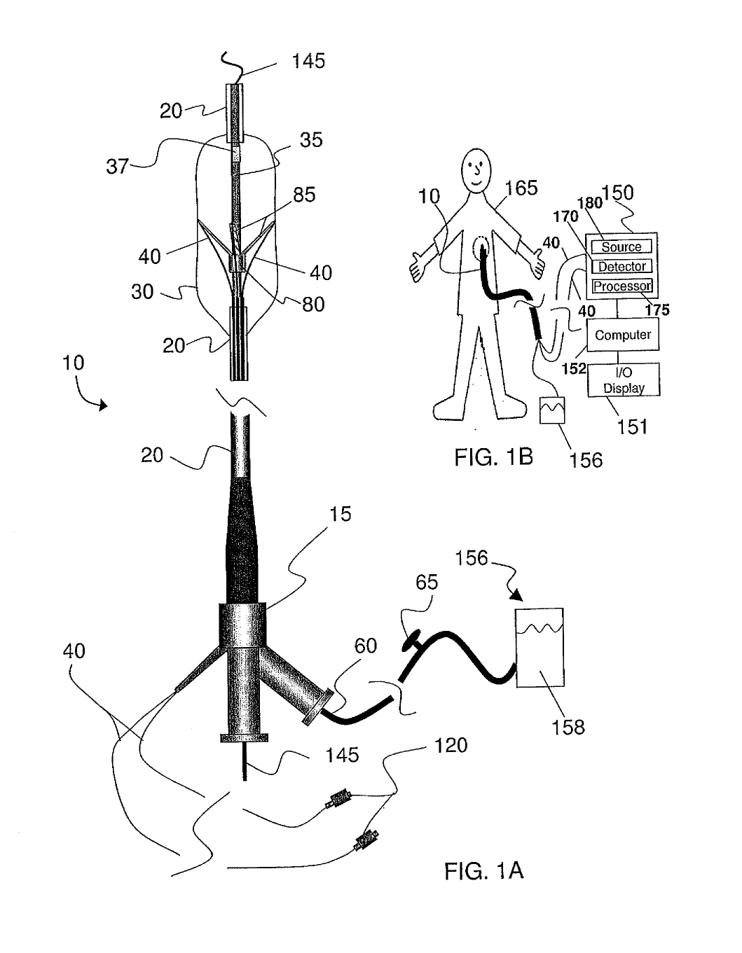

FIG. 1A is an illustrative view of a catheter instrument for analyzing and medically treating a lumen, in accordance with embodiments of the present inventive concepts. FIG. 1B is a block diagram illustrating an instrument deployed for analyzing and medically treating the lumen of a patient, in accordance with embodiments of the present inventive concepts. A catheter assembly 10 can comprise a junction 15 that is connected to a proximal end of a catheter sheath 20 and a balloon 30 that is connected to a distal end of the catheter sheath 20. In an embodiment, the balloon 30 can function as a lumen-expanding balloon, such as, an angioplasty balloon.

The catheter assembly 10 further comprises a guidewire sheath 35 and guidewire 145. The guidewire sheath 35 provides a lumen that allows the catheter assembly 10 to be deployed over a guidewire 145 already deployed within a patient.

The catheter assembly 10 further comprises at least two fibers 40, which can include one or more delivery fiber(s) connected to at least one source 180 and one or more collection fiber(s) connected to at least one detector 170. In an embodiment, the catheter assembly 10 includes two fibers 40, including one delivery fiber and one collection fiber. In another embodiment, the catheter assembly 10 includes four fibers 40, including two delivery fibers and two collection fibers. In another embodiment, the catheter assembly 10 includes four fibers 40, including a first pair of delivery and collection fibers and a second pair of delivery and collection fibers.

The catheter assembly can further comprise a whisker body 80 having a plurality of flexible whiskers 85 that is positioned within the balloon 30. In this embodiment, proximal ends of the whiskers 85 are connected to the whisker body 80 and distal ends of whiskers 85 are attached to tips of fibers 40 so that when the balloon 30 is expanded, the tips of fibers 40 are held against the inner surface of the balloon 30. In an embodiment, the number of whiskers 85 corresponds to the number of fibers 40 provided with the catheter assembly 10.

In an embodiment, the whiskers 85 are manufactured out of a flexible, elastic material and in a manner so as to be pre-disposed to extending radially outward to at least the maximum diameter of an expanded balloon 30. The whiskers 85 are constructed so as to be extremely thin and flexible (material) so as to easily conform to attributes of a surrounding lumen.

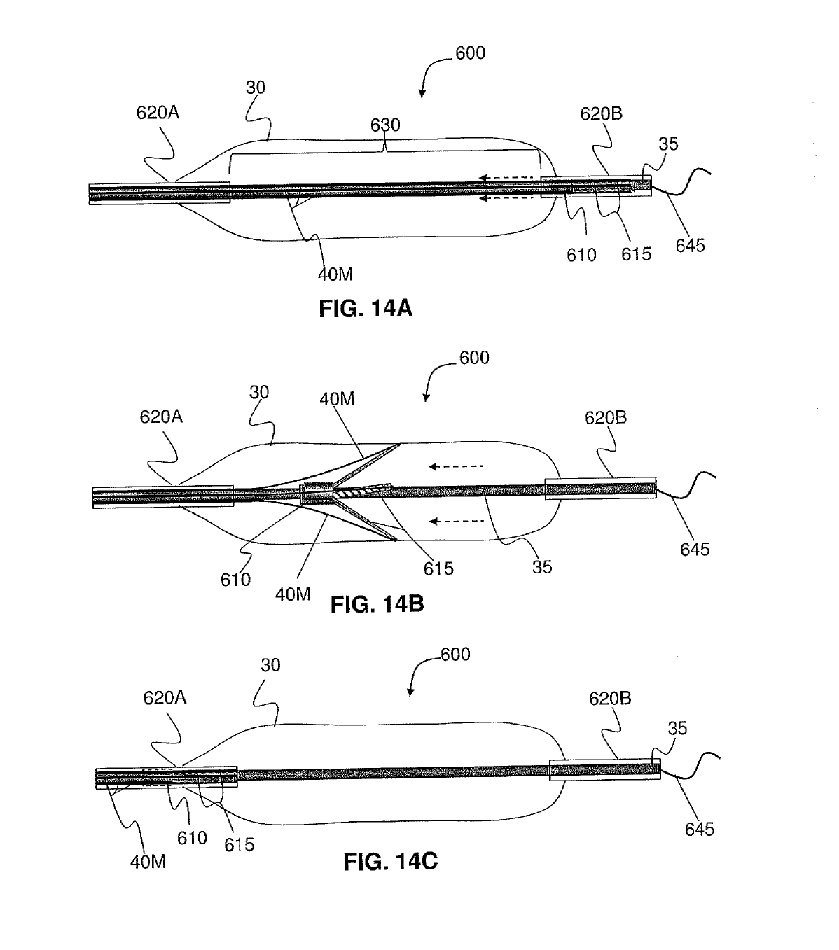

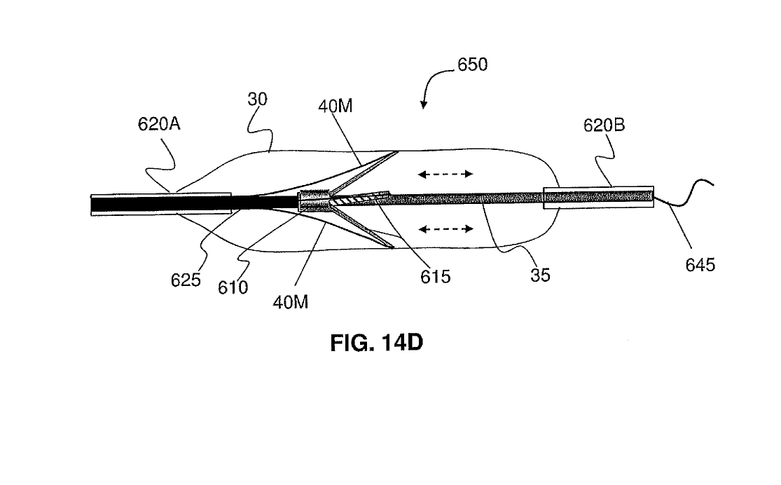

In an embodiment, the whiskers 85 have a width (orthogonal to catheter's longitudinal and radial axis to the whisker) of about 0.012 inches. In other embodiments, the width of the whiskers 85 can be less than about 0.012 inches or greater than about 0.012 inches. In other embodiments, the width of the whiskers 85 can range between about 0.0008 inches to about 0.016 inches. Further, in an embodiment, the whiskers 85 have a length (parallel to the catheter's longitudinal and radial axis to the whisker) of about 2 mm or less. Further embodiments are described below in reference to FIGS. 14A-14F

The whisker body 80 and the whiskers 85 can be constructed of a thermoplastic, such as, polyether ether ketone (“PEEK”) or other thermoplastics. The whisker body 80 and the whiskers 85 can also be constructed of a metal alloy, such as, nitinol or other similar alloys. In an embodiment, the whiskers 85 are constructed of PEEK and have a thickness (along the catheter's radial axis to the whisker) of about 0.005 inches. In other embodiments, the thickness of the PEEK whiskers can range between about 0.003 inches to about 0.01 inches. In another embodiment, the whiskers 85 are constructed of nitinol and have a thickness of about 0.002 inches. In other embodiments, the thickness of the nitinol whiskers 85 can range between about 0.001 inches to about 0.003 inches.

In an embodiment, the whiskers 85 have an outward biasing spring force, which causes the whiskers 85 to expand outward upon inflation of the balloon 30. In an embodiment, after deployment (e.g., expansion) and use of the whiskers 85 within a lumen, the whiskers 85 can be retracted by applying a vacuum pressure to the balloon 30 so that the balloon 30 deflates and subsequently retracts the whiskers 85.

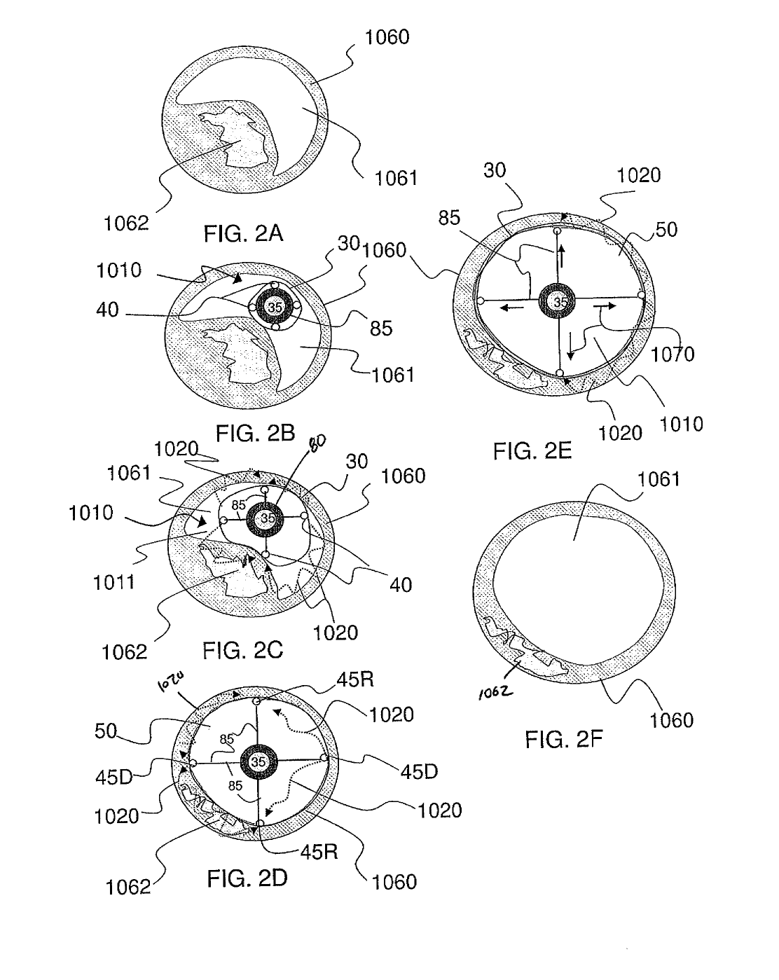

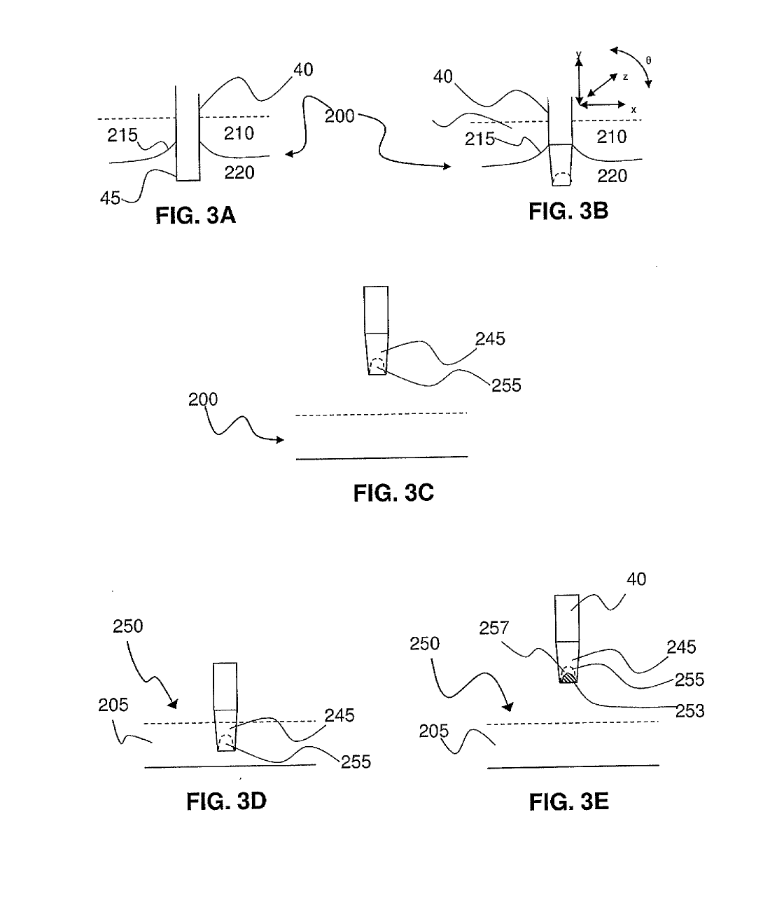

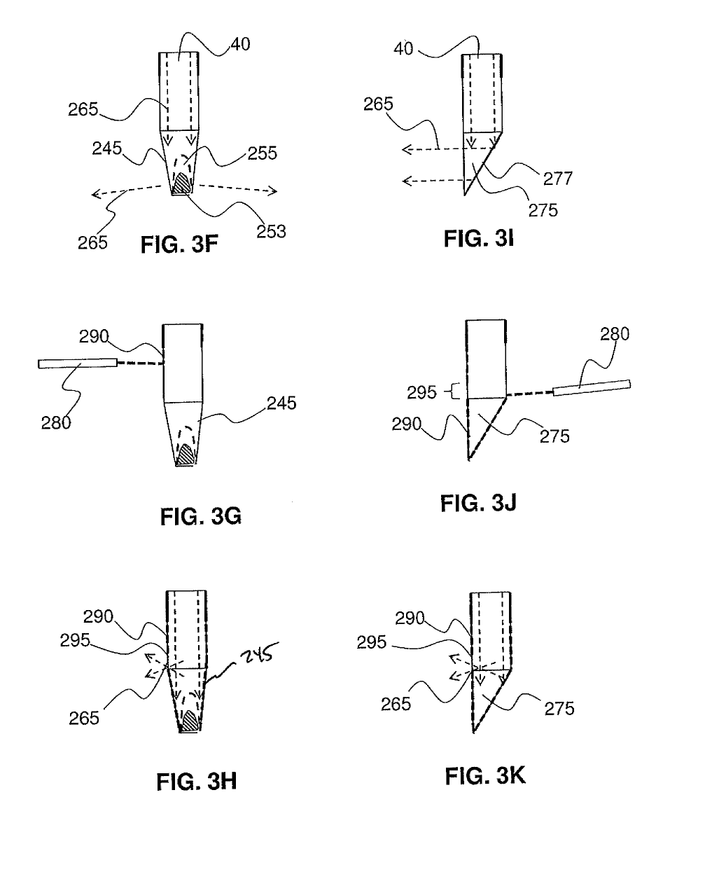

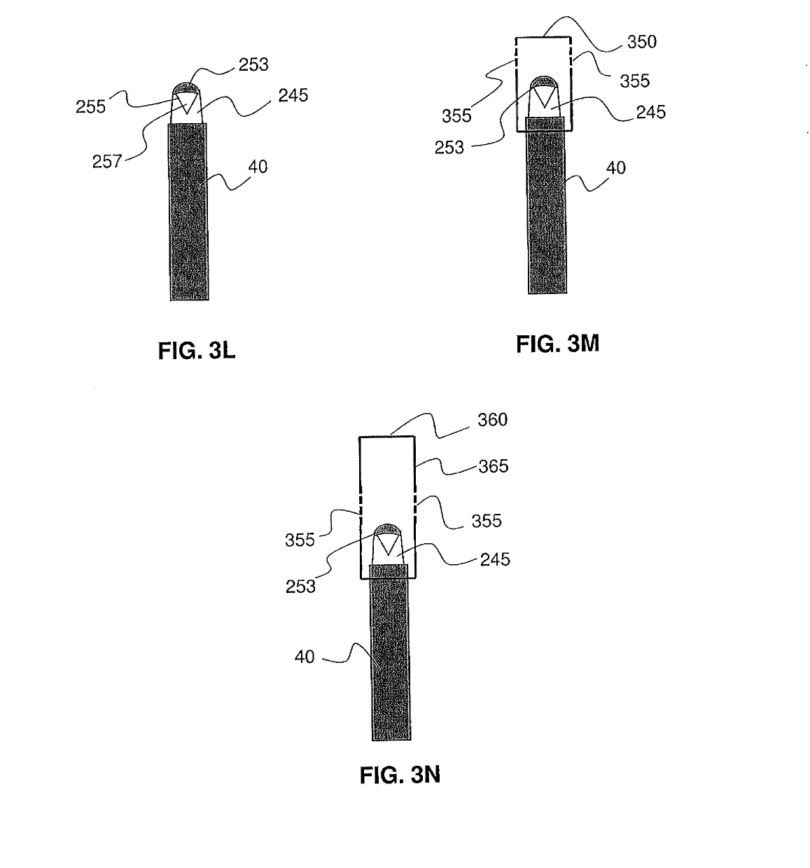

The balloon 30 can comprise a material that is translucent to radiation delivered and collected by the fibers 40, such as, for example, translucent nylon or other translucent polymers. Referring to FIG. 2D, delivery and collection ends 45 of the fibers 40 are preferably configured to deliver and collect light about a wide angle, such as, for example, between about at least a 120 to 180 degree cone around the circumference of each fiber, directed radially outward from about the center of the catheter 10. Various methods for forming such delivery and collection ends are described in more detail herein (e.g., see FIGS. 3A-3E and accompanying description herein). Various embodiments in accordance with the present inventive concepts allow for diffusely reflected light to be readily delivered and collected between the fibers 40 and the tissue surrounding the catheter 10.

Referring back to FIGS. 1A and 1B, a proximal end of the balloon catheter assembly 10 includes a junction 15 that distributes various conduits within the catheter sheath 20 to external system components. The fibers 40 can be fitted with connectors 120 (e.g. FC/PC type) compatible for use with light sources, detectors, and/or analyzing devices such as spectrometers. Two radiopaque marker bands 37 are fixed about guidewire sheath 35 in order to help an operator to obtain information about the location of catheter 10 in the body of a patient (e.g. with the aid of a fluoroscope).

The proximate ends of fibers 40 are connected to a light source 180 and/or a detector 170 (which are shown integrated with an analyzer/processor system 150). The analyzer/processor system 150 can comprise, for example, a spectrometer which includes a processor 175 for processing/analyzing data received through the fibers 40. A computer 152 can be connected to the analyzer/processor system 150, which can provide an interface for operating the instrument 200. The computer 152 can further process spectroscopic data (including, for example, through chemometric analysis) in order to diagnose and/or treat the condition of a subject 165. Input/output components (I/O) and viewing components 151 are provided in order to communicate information between, for example, storage and/or network devices and the like and to allow operators to view information related to the operation of the instrument 10.

Various embodiments comprise an analyzer/processor system 150, for example, including a spectrometer, that is configured to perform spectroscopic analysis within a wavelength range between about 250 nanometers and about 2500 nanometers. The various embodiments can include embodiments configured to perform spectroscopic analysis in the near-infrared spectrum between about 750 nanometers and about 2500 nanometers. Further, embodiments can be configured for performing spectroscopy within one or more subranges that include, for example, about 250 nanometers to about 930 nanometers, about 1100 nanometers to about 1385 nanometers, about 1550 nanometers to about 1850 nanometers, and about 2100 nanometers to about 2500 nanometers. Various embodiments are further described in, for example, related applications U.S. application Ser. No. 11/537,258, filed on Sep. 29, 2006, titled “SYSTEMS AND METHODS FOR ANALYSIS AND TREATMENT OF A BODY LUMEN”, and U.S. application Ser. No. 11/834,096, filed Aug. 6, 2007, titled “MULTI-FACETED OPTICAL REFLECTOR”, the entire contents of each application being herein incorporated by reference.

The junction 15 can comprise a flushing port 60 for supplying or removing fluid media (e.g., liquid/gas) 158, which can be used to expand or contract the balloon 30. Fluid media 158 is held in a tank 156 from which it is pumped in or removed from the balloon(s) 30 in response to the actuation of a knob 65. Fluid media 158 can alternatively be pumped into or out of the balloon(s) 30 with the use of automated components (e.g. switches/compressors/vacuums). Solutions for expansion of the balloon are preferably non-toxic to humans (e.g. saline solution) and are substantially translucent to the selected light radiation.

FIGS. 2A-2F are cross-sectional views illustrating the sequential steps of performing a balloon angioplasty procedure, in accordance with embodiments of the present inventive concepts. FIG. 2A is a cross-sectional view of a constricted body lumen 1061 having a lumen wall 1060. The lumen 1061 may be constricted due to a blockage, for example, a blockage 1062 caused by an accumulation of lipid content.

As shown in FIG. 2B, a balloon catheter 1010, for example of various embodiments described herein, is inserted into the constricted lumen 1061 in accordance with conventional procedures. In an embodiment, the balloon catheter 1010 comprises a guidewire sheath 35, a balloon 30, at least one delivery fiber 40, at least one collection fiber 40, a whisker body 80 and whisker arms 85. In a treatment procedure according to an embodiment of the present inventive concepts, a physician first inserts a guidewire 145 (shown in FIG. 1A) into the constricted body lumen 1061 of a patient via a puncture point, such as, for example, a puncture point located at the groin or wrist of a patient. Next, the physician places the balloon catheter 1010 on the guidewire 145 and positions the balloon catheter 1010 within the constricted body lumen 1061 of the patient. The balloon catheter 1010 comprises a balloon 30 and whiskers 85 within balloon 30 that are, upon entry to the constricted lumen 1061, in an unexpanded state.

As shown in FIG. 2C, the positioned balloon catheter 1010 is partially inflated by delivering fluid, such as, a gas or liquid, through a port of the balloon catheter 1010 and into the balloon 30 of the balloon catheter 1010 (as further described in reference to various embodiments herein). The balloon catheter 1010 comprising at least one delivery fiber 40 and at least one collection fiber 40 positioned against the inner wall of balloon 30 enables the collection of data of the spectral features of the lumen wall 1060 by delivering optical radiation 1020 from a delivery fiber 40 to the lumen wall 1060, and collecting optical radiation 1020 that is reflected from the lumen wall 1060 and received by a collection fiber 40. The collection of data of the spectral features of the lumen wall 1060 can be used to determine the position of the balloon catheter 1010 with respect to a target region of the constricted body lumen 1061. Since the lumen wall information is obtained via spectral analysis in real-time, the physician can rely on this information to determine the relative position and type of diseased area or blockage 1062 of the lumen 1061, and, accordingly, can help a physician determine the necessary procedure (e.g. balloon angioplasty, stent insertion) and/or type of stent, bypass, and/or systemic drug therapy that may be best for the patient. The physician or operator can decide, for example, to cease inflation of the balloon 30 and withdraw the catheter 1010 from the patient based on signals corresponding to the optical radiation 1020 reflected from the lumen wall 1060, which are, for example, indicative of a lesion highly prone to rupture.

In addition, signals corresponding to the optical radiation 1020 can be used to more properly control the rate of inflation of the balloon catheter 1010 and the maximum inflation of the balloon catheter 1010. As such, the physician or operator can gradually inflate the balloon catheter 1010 while the system monitors the signals corresponding to the optical radiation 1020 reflected from the lumen wall 1060, which can detect the presence of blood and the proximity of the vessel wall 1060 to the balloon wall 30. In addition, signals can be measured for the presence of inflation media. If a relatively significant level of blood is detected about the entire periphery of catheter 1010 and outer covering of the balloon 30, it can be determined that the balloon catheter 1010 is not likely sufficiently expanded for its applicable purpose (e.g., angioplasty, pre-stenting dilation, stent deployment, and/or post-stenting expansion). When the signal for blood has substantially diminished, the operator can further controllably inflate the catheter 1010 to an appropriate level.

In an embodiment, diffuse reflectance spectroscopy is employed between wavelengths of about 250 nanometers to about 2500 nanometers. In an embodiment, ratios between the absorbance signals of two or more wavelengths are used to indicate a relative proximity of the balloon surface to a lumen wall 1060. In an embodiment, one of the two or more wavelengths is between about 250 nanometers and about 750 nanometers and another of the two or more wavelengths is between about 800 nanometers and about 1000 nanometers. In an embodiment, one of the two or more primary wavelengths for detecting the presence of blood apart from balloon inflation media is green visible light (or about 520 nanometers) and one of the two or more secondary or reference wavelengths is about between about 800 to 1000 nm, 1300 nm and 1350 nm, between about 1380 and 1450 nm, and between about 1550 nm and 1850 nm which are generally less sensitive to changes in the presence of blood than, for example, green light. Other wavelengths, including more specific wavelengths of 1450 and/or 1550 nm, will generally be more sensitive to changes in the presence of water and/or blood for purposes of various described embodiments such as for detecting the amount of balloon media and blood present. In an embodiment, a ratio between a primary wavelength (sensitive to change in the targeted characteristic) and a reference wavelength (substantially less sensitive to change in the targeted characteristic) can be calculated in order to remove anomalies in the readings relating to, for example, noise and differences between catheters. In an embodiment, a ratio of absorption between the amount of absorption of at least one primary radiation signal and an amount of absorption of at least one reference radiation signal can be measured and calculated in order to remove anomalies in the readings relating to, for example, noise and differences between catheters.

In another embodiment, spectroscopy is employed with one or more wavelengths with predetermined spectra profiles known to have at least nominally predictable relationships with the content of adjacent blood alone or tissue and/or balloon inflation media. In an embodiment, one or more primary wavelengths selected from 407 nanometers, 532 nanometers, and a reference wavelength is selected between about 800 nanometers and about 1000 nanometers are spectroscopically analyzed. In an embodiment, diffuse reflectance spectroscopy is used. In an embodiment, previously measured ratios between two or more of these wavelengths at various blood and/or balloon media depths are programmed into a system, and later compared to in-process data collected during an actual procedure. In an embodiment, the one or more wavelengths consist of wavelengths of about 532 nanometers and about 407 nanometers and in another embodiment consist of about 532 nanometers and about 800 nanometers.

In another embodiment, the relative level of inflation of the balloon 30 is determined by measuring the amount of absorption of a radiation signal across the balloon media between at least one delivery and at least one collection fiber. In an embodiment, two or more radiation signals having different wavelengths are measured between the at least one delivery fiber and the at least one collection fiber. In an embodiment, at least one of the radiation signals, a primary radiation signal (having a primary wavelength or range of wavelengths), is generally more sensitive to a change in the presence of water and/or blood such as one of the wavelengths described above including, for example, 1550 nanometers and at least one of the radiation signals is employed as a reference radiation signal (having a reference wavelength or range of wavelengths) where its change in absorption in water compared to the primary wavelength is relatively insignificant over short distances (e.g., over 4 mm or less) such as, for example, a reference wavelength of about 1310 nanometers when used with a primary wavelength of 1550 nanometers. In an embodiment, the ratio between the primary wavelength(s) and reference wavelength(s) is calculated and used to compare different levels of expansion of balloon 30.

Generally, typical angioplasty-type procedures rely on inaccurate fluoroscopy measurements and balloon expansion profiles made prior to catheter deployment to determine the level of fluid pressure/inflation needed. In order to avoid risky complications, these traditional procedures often overinflate the balloon catheter. An under-expanded stent, for example, may not only fail to properly support a targeted vessel area but also cause additional undesired blockages itself. Overexpansion, however, presents its own risks (e.g. rupture and other vessel damage) and an angioplasty-type procedure may therefore be avoided altogether as a treatment. Various embodiments of the present inventive concepts as described herein can help avoid these occurrences by more accurately determining apposition of the catheter balloon against a vessel wall in real-time. Accordingly, apposition of the catheter balloon against a vessel wall can be determined during an angioplasty-type procedure, while the balloon catheter is positioned within a patient.

A signal corresponding to the optical radiation 1020 indicative of the presence of blood about only portions of catheter 1010 could also be used to help determine, for example, the presence and peripheral location of a hard (e.g., calcified) lesion. If the localized presence of blood is detected when the balloon should be substantially apposed to lumen wall 1060, the signals may be indicative of a deformed mal-apposed balloon that may result when such hard lesions significantly resist expansion while other portions of the vessel do not so resist. Under these circumstances, the mal-apposed balloon may either trap blood in pockets between the balloon wall and the vessel wall or allow blood to freely flow by along certain portions of the balloon. Signals corresponding to the optical radiation 1020 could further verify the presence of, for example, such elements as calcium or other elements indicative of hard lesions. Since an embodiment of the present inventive concepts can also identify weaknesses along the lumen wall 1060 prior to fully deploying an angioplasty balloon 30 at a target region of the lumen wall 1060, the embodiments can reduce the risk of a rupture occurring at or near the blockage 1062 during or after an angioplasty procedure.

As shown in FIG. 2D, the catheter 1010 is shown further inflated and whiskers 85 and fibers 40 substantially apposed to lumen 1061 at the target region for treatment (e.g., balloon angioplasty and/or stent insertion (stent not shown)). Optical radiation 1020 is transmitted from a delivery fiber tip 45D and transmitted through the balloon catheter 1010 to the catheter surface that abuts the lumen wall 1060. The optical radiation 1020 passes through the surface of the balloon 30 and impinges the target region of the lumen wall 1060 and can interact with the tissue/fluids therein in the manner of, for example, fluorescence, luminescence, and/or diffuse reflectance as described in detail herein. Collection fibers tips 45R can receive the emitted optical radiation from the lumen wall 1060 and transfer them to one or more detectors and for further processing (e.g., a spectroscopic analysis system). In order to separately process and assess signals from a particular circumferential portion of a lumen 1060, an embodiment activates, e.g., supplies light to, delivery fiber tip(s) 45D while other delivery fiber(s) are deactivated by the system. Since the balloon catheter 1020 is in direct contact with the lumen wall, such that little or no blood is between the balloon and the lumen wall, high-quality spectral data can be obtained. This additional spectral data allows the physician to receive in real-time the treatment results, as well as current physiological and pathological changes on the treatment.

For example, if a lumen is being inspected in an angioplasty application (e.g., pre-dilation, stenting, post-dilation), the physician can rapidly make a decision for subsequent therapy, e.g., a stent insertion and/or a drug local injection therapy after a sample balloon angioplasty for second treatment. The spectral data can also indicate the preferred stent to be selected for treatment, of any required future treatment, etc. by analyzing pathology results on the lumen wall. The spectral data can also be stored for future analysis or comparison to current treatment(s). In an embodiment, at the point when the catheter 1020 substantially apposes the lumen wall 1060 (e.g., as shown in FIG. 3D), the physician can use the balloon's expansion profile and collected data to determine whether and how much further to inflate the balloon catheter for an applicable treatment.

In an embodiment, selected drugs (not shown) are coated over the outside of the balloon 30 of the balloon catheter 1010. In an embodiment, one or more of the drugs coating the balloon 30 can be activated, e.g., so as to provide therapeutic effect, by the emission of selected radiation wavelengths from fiber ends 45 to the balloon 30 at various stages of the deployment of the catheter 1010. A physician, for example, can use information gathered from prior analysis performed by a balloon catheter 1010 to decide whether and if selected drugs should be activated or left inactivated.

As shown in FIG. 2E, the balloon catheter 1010 is further inflated in the direction of arrows 1070 and is shown dilating the lumen 1060 as in, for example, an angioplasty. Further data can be collected through the fiber optical system in order to monitor and assess the ongoing treatment. The treated and analyzed lumen 1060 is shown in FIG. 3F after deflation and removal of balloon catheter 1010.

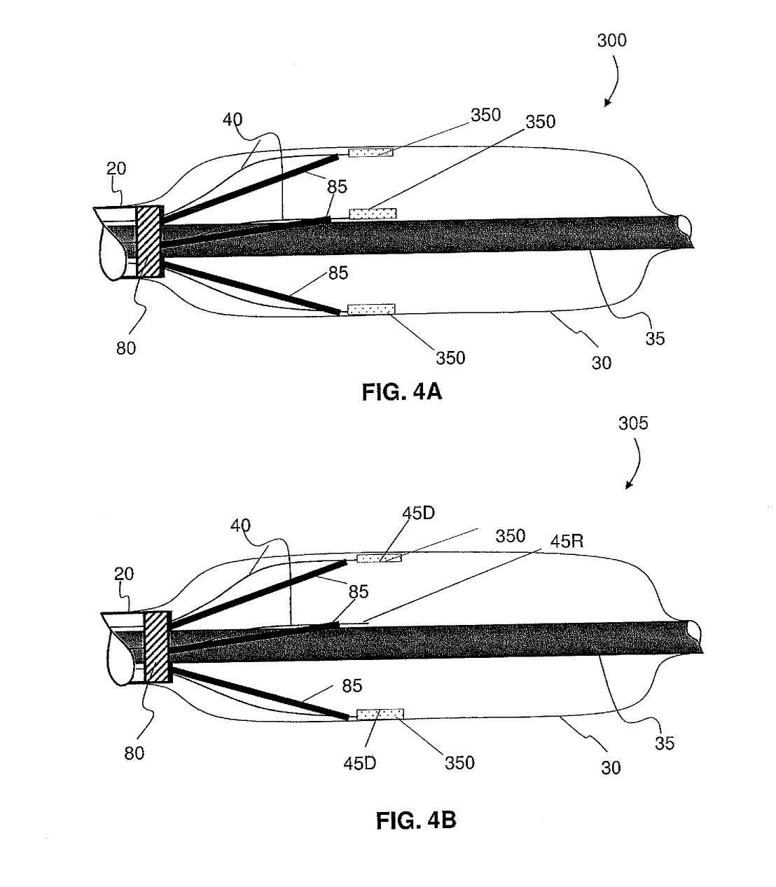

FIG. 3A is an illustrative schematic view of a fiber tip being formed in an etchant solution in a method, in accordance with embodiments of the present inventive concepts. FIG. 3B is an illustrative view of the fiber tip of FIG. 3A, while placed in an etchant solution, in accordance with embodiments of the present inventive concepts. FIG. 3C is an illustrative schematic view of the fiber tip of FIG. 4A after extraction from an etchant solution, in accordance with embodiments of the present inventive concepts. FIG. 3D is an illustrative schematic view of a recessed fiber tip being placed in a sealant solution, in accordance with embodiments of the present inventive concepts. FIG. 3E is an illustrative schematic view of the fiber tip of FIG. 3D after extraction from the sealant solution of FIG. 3D, in accordance with embodiments of the present inventive concepts. The etching of the fiber end in the manner described herein permits radiation or collection of radiated signals in directions substantially perpendicular to the longitudinal axis of the fiber's tip. This feature supports various preferred embodiments of fibers connected to elongate arms (whiskers) as described herein that rely on such off-axis delivery or collection.

In an embodiment, the process for forming a fiber tip 245 occurs (as shown in FIG. 3A) by placing the end 45 of a fiber 40 in a bath 200 including an etchant 220. An organic solvent 210 (e.g., silicone) can be included in the bath so as to control formation of a meniscus 215 and to prevent inadvertent exposure of portions of fiber 40 to the etchant 220. Depending on the fiber type and the desired profile/shape of tip 245, the fiber 40 is held in the bath 200 of etchant solution 220 for a predetermined amount of time. In an embodiment, the fiber 40 has a graded-index core with a diameter of between about 50 microns and about 100 microns, and is held in an etchant solution 220 comprising Hydrofluoric acid (HF) for a period between about 4 minutes to about 15 minutes or more.

Referring to FIG. 3B, the fiber 40 can also be moved and repositioned in the etchant 220 to affect the shape of tip 245.

Referring to FIG. 3C, the etchant solution 220 gradually removes material from the cladding/core interior of the end 45 of the fiber to form a fiber tip 245 having a shaped recess 255 within the cladding/core interior of the fiber 40. Methods for shaping fiber tips in this manner are more fully described in U.S. Provisional Application No. 61/025,514, filed Feb. 1, 2008, titled “SHAPED FIBER ENDS AND METHODS OF MAKING SAME”, PCT Application No. PCT/US2009/044078, filed on May 15, 2009, titled “SHAPED FIBER ENDS AND METHODS OF MAKING SAME”, and U.S. Provisional Application No. 61/082,721, filed Jul. 22, 2008, titled “SYSTEMS AND METHODS FOR ANALYSIS AND TREATMENT OF A BODY LUMEN”, the entire contents of each application being herein incorporated by reference.

Referring in particular to FIGS. 3D and 3E, a fiber tip 245 with a shaped recess, such as, for example, recess 255 shown in FIG. 3C is placed in a sealant bath 250 of sealant 205 so as to form a protective seal 253 across the opening of the recess and help prevent contaminants including, for example, fluid media from interfering with the optical functions of the fiber tip 245. In on embodiment, the recess 255 is concave.

In various embodiments, sealants for use in protecting the recess 255 include, for example, pyroxylin, thermoplastics such as ethylene-vinyl acetate, and thermosetting plastics such as ultraviolet cured glass glue. In an embodiment, a Loctite® brand series 3345 sealant, by Henkel Corporation, Henkelstraβe 67, 40191 Dusseldorf, Germany, or other similar type sealant is used to protect the recess 255.

Referring to FIG. 3E, after the tip 245 is extracted from sealant bath 250, protective seal 253 is formed within recess 255. In an embodiment, an air gap 257 may be formed between the protective seal 253 and the surface of recess 255. Air gap 257 can, for example, aid in directing refracted light incident upon the recess 255 toward directions oblique to the longitudinal axis of fiber tip 245 (see, e.g., sample signal trace lines 265 of FIGS. 3F-3K).

Various other delivery and collection end arrangements of fibers 40 can be adapted for use in embodiments of the present inventive concepts, such as, for example, those arrangements described in co-pending and related U.S. patent application Ser. No. 11/537,258, filed on Sep. 29, 2006, published as Patent Application Publication No. 2007/0078500 A1, the entire contents of which is incorporated herein by reference.

In embodiments, the recess 255 can have other shapes, such that a vertex is located within the core of the tip. In other embodiments, recess 255 can have other shapes that comprise higher order polynomial curves. In other embodiments, the recess has a curved surface, the curved surface having a vertex within the core.

FIG. 3F is an illustrative schematic view of the fiber tip of FIG. 3E with sample signal trace lines 265, in accordance with embodiments of the present inventive concepts. A portion of the light delivered through fiber 40 that is incident upon the surface of the recess 255 will be reflected at angles oblique to the longitudinal direction of the fiber. Some light will also be incident upon and reflect off of protective seal 253, helping direct additional light in directions oblique to the longitudinal axis of the fiber. Light directed at the tip of fiber 40 from oblique angles will likewise be collected by fiber 40.

FIG. 3G is an illustrative view of a reflective coating 290 being applied to the fiber tip of FIG. 3F by an applicator 280, in accordance with embodiments of the present inventive concepts. FIG. 3H is an illustrative view of the fiber tip of FIGS. 3F and 3G with sample signal trace lines after application of a reflective coating, in accordance with embodiments of the present inventive concepts. A side section of the tip is left uncoated, allowing light to travel in or out of the opening. The light that travels in or out of the opening will be dispersed more diffusely than the more coherent transmission profiles of the examples shown in FIG. 3F or 3I without discrete openings 295. The coating can be applied using a number of materials and methods, including, in an embodiment, reflective metallic materials, such as, gold, silver, platinum, and the like, which can be applied with the use of ion-assisted deposition and/or sputtering techniques. Reflective inks or sprays can also be applied, after which the opening 295 can be cleared with a laser. The opening 295 can be formed around the circumference of the fiber tip 245 or, in an embodiment, just around a portion of the fiber tip 245 so as to direct most of the light to or from a preferred direction.

FIG. 3I is an illustrative schematic view of a side-fire type of fiber optic tip, in accordance with embodiments of the present inventive concepts. The tip 275 of the fiber 40 is cleaved at an oblique angle and a reflective coating 277 is applied to the angled edge so as to direct light to or from fiber 40 at an oblique angle.

FIG. 3J is an illustrative view of an additional reflective coating 280 being applied to the fiber tip of FIG. 3I so as to form a discrete opening 295 by an applicator 280. In similar fashion as exemplified in FIG. 3H, the opening 295 primarily allows external light transmission that has been reflected substantially about the tip area 275 prior to exiting, creating a more diffuse pattern of transmission.

FIG. 3K is an illustrative view of the fiber tip of FIGS. 3I and 3J with sample signal trace lines after application of a reflective coating, in accordance with embodiments of the present inventive concepts.

FIG. 3L is an illustrative view of a fiber tip with an etched recess, in accordance with embodiments of the present inventive concepts. FIG. 3M is an illustrative view of the fiber tip of FIG. 3L with a light diffusing covering, in accordance with embodiments of the present inventive concepts. In an embodiment, a fiber tip 245 includes recess 255, a cap 253 and an air gap 257.

In an embodiment, as shown in FIG. 3M, the fiber tip 245 includes a diffusing covering 350 that surrounds the cap 253 and extends beyond cap 253. In an embodiment, the diffusing covering 350 completely surrounds the tip 245.

In an embodiment, the diffusing covering 350 is coated with a reflective material with the exception of a circumferential window 355 that allows light to be passed through the covering for distribution or collection. In an embodiment, the diffusing covering 350 comprises PEEK, which provides light-diffusing properties. In an embodiment, the reflective material comprises a thin metallic layer, such as, gold, silver, platinum or other like material. In an embodiment, the metallic layer is applied through the process of ion-assisted deposition.

In an embodiment, a PEEK covering around fiber tip 245 has a radial distance from the external surface of the tip of between about 0.001 inches and about 0.01 inches and preferably of about 0.003 inches. In an embodiment, the longitudinal length of the PEEK covering is between about 1.2 millimeters and about 1.5 millimeters with the fiber tip extending through approximately about 0.5 millimeters to about 0.75 millimeters of the length of the PEEK.

FIG. 3N is an illustrative view of the fiber tip of FIG. 3L with a light diffusing tip 360, in accordance with embodiments of the present inventive concepts. A light diffusing tip 360 includes a section 365 that extends beyond window 355 and is also coated with a reflective material. This extended section 365 allows for further diffusion of light prior to its passage out of the window 355 or transmission through the fiber 40 for collection. In an embodiment, the diffusing covering 360 comprises PEEK and extends about 2 millimeters in length with the section 365 and the window 355, each extending about a third of the total length of the covering 360.

FIG. 4A is an expanded illustrative view of the treatment end of a catheter instrument 300, in accordance with embodiments of the present inventive concepts. In an embodiment, each of the ends of the fibers 40 includes a diffusing covering 350. This allows for the distribution and collection of light about a wide angle.

FIG. 4B is an expanded illustrative view of the treatment end of a catheter instrument 305, in accordance with embodiments of the present inventive concepts. In an embodiment, the delivery fiber tips 45D include a diffusing covering 350 and the collection fiber tips 45R do not have diffusing coverings so as to improve the amount of light that is collected.

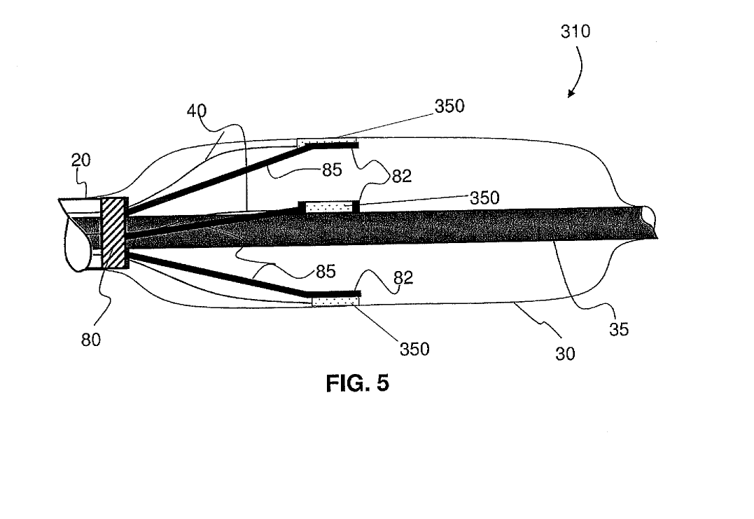

FIG. 5 is an expanded illustrative view of the treatment end of a catheter instrument, in accordance with embodiments of the present inventive concepts. In an embodiment, the whiskers 85 include reflective ends 82 with a reflective surface directed outwardly from the catheter 310 so as to enhance the delivery or collection of radiation traveling toward the reflective surfaces from locations external to balloon 30. The surfaces can include a reflective coating comprising reflective materials, such as, gold, silver, platinum or like materials. The reflective coating can further comprise other reflective particles deposited on its surface. In an embodiment, the reflective ends 82 can be positioned between the inner surface of balloon 30 and the ends of fibers 40 so as to enhance delivery or collection of radiation directed within balloon 30. For example, such an embodiment can be used to measure absorption of light traveling within balloon 30 from a delivery fiber to a collection fiber.

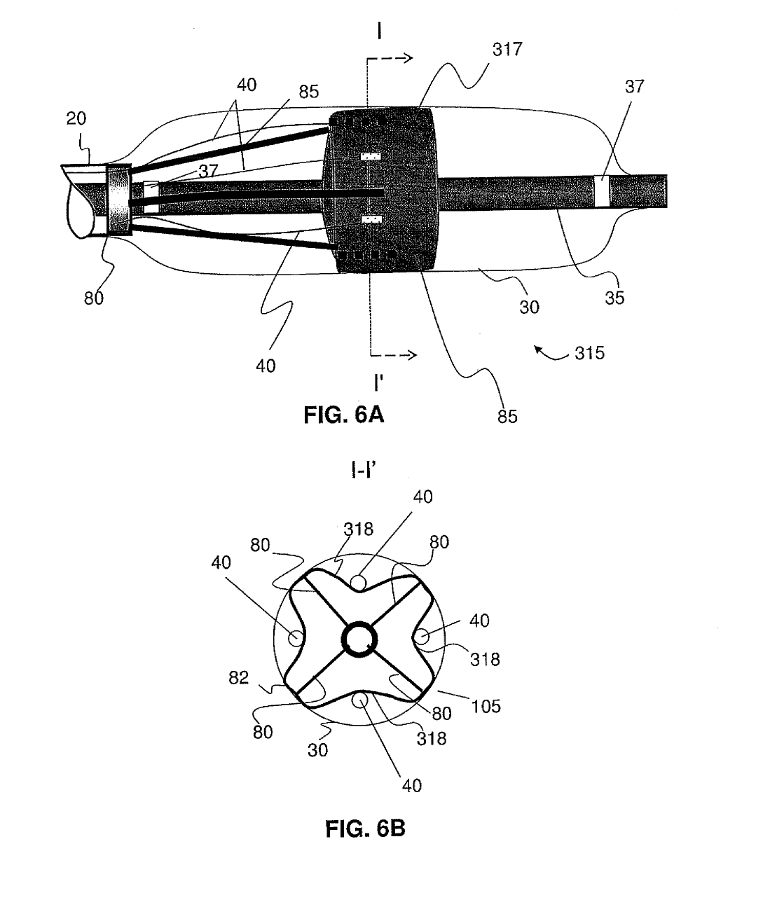

FIG. 6A is an expanded illustrative view of the treatment end of a catheter instrument 315, in accordance with embodiments of the present inventive concepts. FIG. 6B is a cross-sectional view of the catheter of FIG. 6A, taken along section lines I-I′ of FIG. 6A, in accordance with embodiments of the present inventive concepts. In an embodiment, a reflective surface 317 extends within the inner perimeter of the balloon 30, promoting delivery and collection of signals external to balloon 30. In an embodiment, the whiskers 85 push portions of reflective surface 317 against the inner wall of balloon 30. When the whiskers 85 push the reflective surface 317 outwardly, the ends of fibers 40 are subsequently pushed outwardly as well from within a reflective pocket 318 of reflective surface 317 as shown in FIG. 6B.

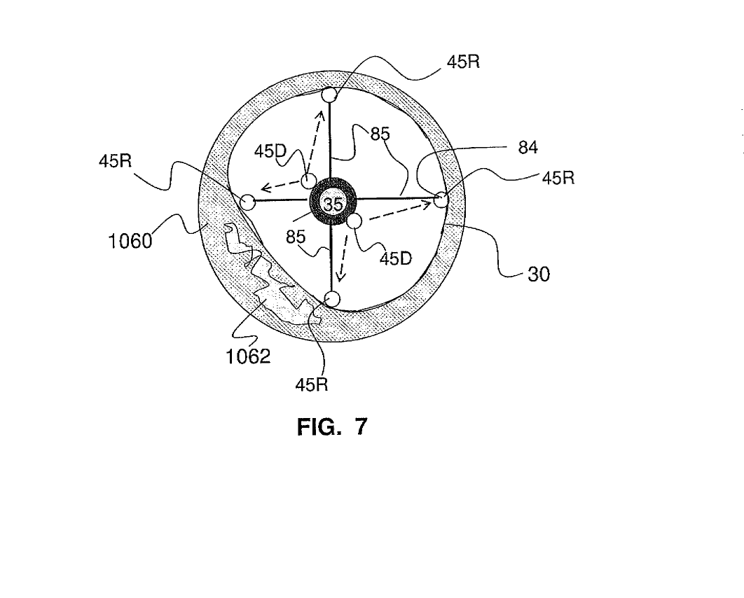

FIG. 7 is an expanded illustrative cross-sectional view of the treatment end of a catheter instrument, in accordance with embodiments of the present inventive concepts. In addition to fibers attached to the whiskers 85, such as, in accordance with the embodiment of FIG. 1A, two additional fibers are attached to the guidewire lumen 35. This arrangement allows for a shorter signal path of travel between a delivery fiber (e.g., through fiber tip 45D) and a collection fiber (e.g., through a fiber tip 45R).

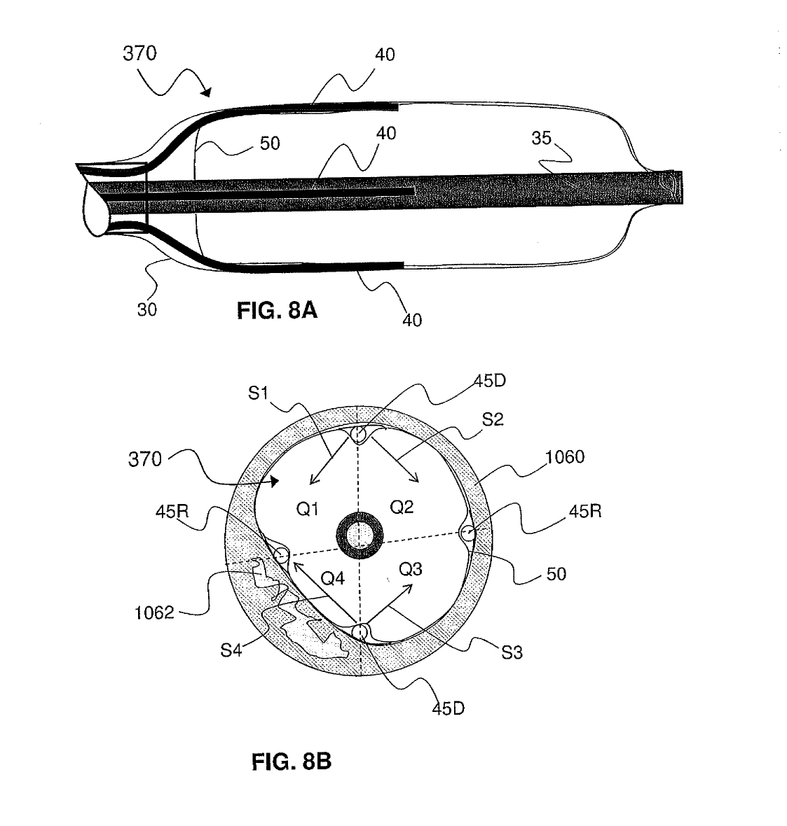

FIG. 8A is an illustrative schematic of a catheter configuration 370 including two delivery fibers and two collection fibers positioned along the inner surface of a balloon 30, in accordance with embodiments of the present inventive concepts. FIG. 8B is an illustrative cross-sectional schematic of the delivery fibers and collection fibers positioned for analyzing the expansion profile of the balloons of FIG. 8A within a lumen, in accordance with embodiments of the present inventive concepts. Two delivery fibers 45D and two collection fibers 45R are positioned along the inner surface of the balloon 30 and along the outside surface of a second balloon 50. Delivery fibers 45D and collection fibers 45R are held between an inner balloon 50 and an outer balloon 30 by the simultaneous expansion of balloons 30 and 50. The two balloons 30 and 50 can be expanded via separate inflation lumens, for example, or other apparatus and methods such as further described in co-pending U.S. patent application Ser. No. 12/350,870, filed Jan. 8, 2009, and published as U.S. Patent Application Publication No. 2009/0187108 A1, the contents of which are herein incorporated by reference in their entirety. The surfaces of inner balloon 50 are translucent to radiation delivered by delivery fibers 45D, allowing signals to travel to collection fibers 45R and be analyzed in order to determine the relative positioning and expansion/under-expansion of each of the circumferential regions Q1-Q4. For example, as illustrated in FIG. 8B, the signals S1 and S4 travelling on the left-hand side of the lumen travel a shorter distance from a delivery fiber to a collection fiber than do the other signals S2 and S3, thus indicating that the circumferential region is under-expanded relative to the other circumferential regions. In addition, the combination of signals can indicate that the entire lumen is under-expanded.

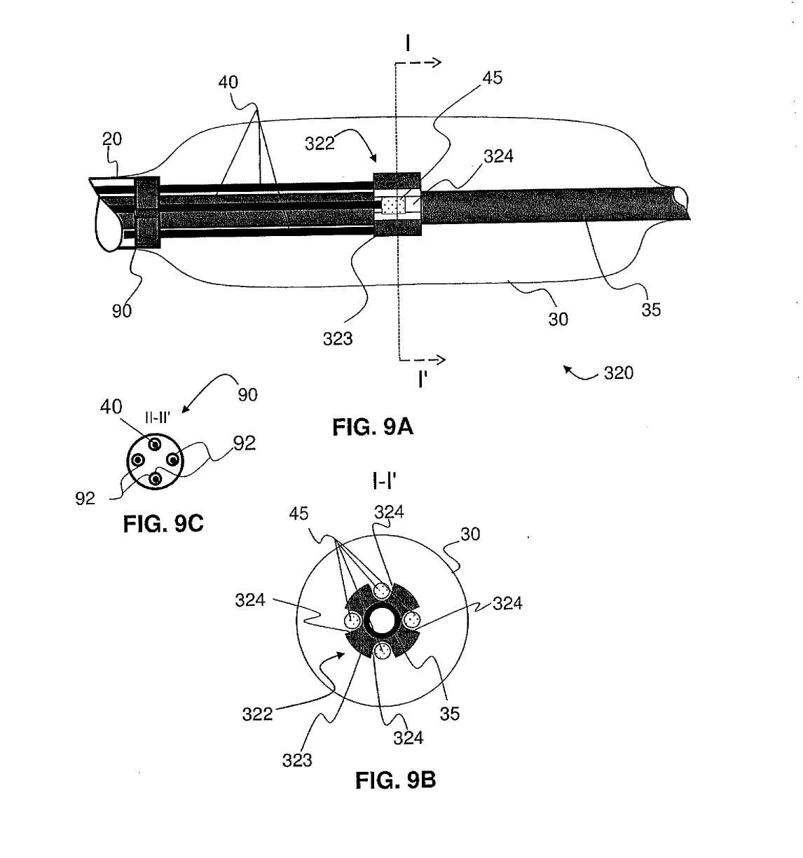

FIG. 9A is an expanded illustrative view of the treatment end of a catheter instrument 320, in accordance with embodiments of the present inventive concepts. FIG. 9B is a cross-sectional view of the catheter of FIG. 9A, taken along section lines I-I′ of FIG. 9A, in accordance with embodiments of the present inventive concepts. FIG. 9C is a cross-sectional view of a fiber alignment ring 90 for aligning fibers 40 passing to a fiber alignment element 324. In this embodiment, fibers 40 are fixed contiguously to the catheter sheath 20 and guidewire lumen 35. A fiber alignment element 322 is formed about guidewire lumen 35 having recesses 324 in which fiber tips 45 can be positioned and aligned therein. In an embodiment, the recesses 324 can have reflective surfaces that can help distribute or collect light to or from an area generally concentrated across an adjacent lumen (not shown). In an embodiment, reflective surfaces are parabolic and shaped to more widely distribute signals toward an adjacent lumen. The shape of the parabola can be optimized based on the size and distribution/collection profile of fiber ends 45 and the estimated distance between distribution/collection ends 45 from each other and from the lumen wall (or the outside of outer balloon 30). Light blocking elements 323 prevent signals from traveling directly from a delivery fiber to a collection fiber without first being diffused about the area adjacent to a delivery fiber tip 45.

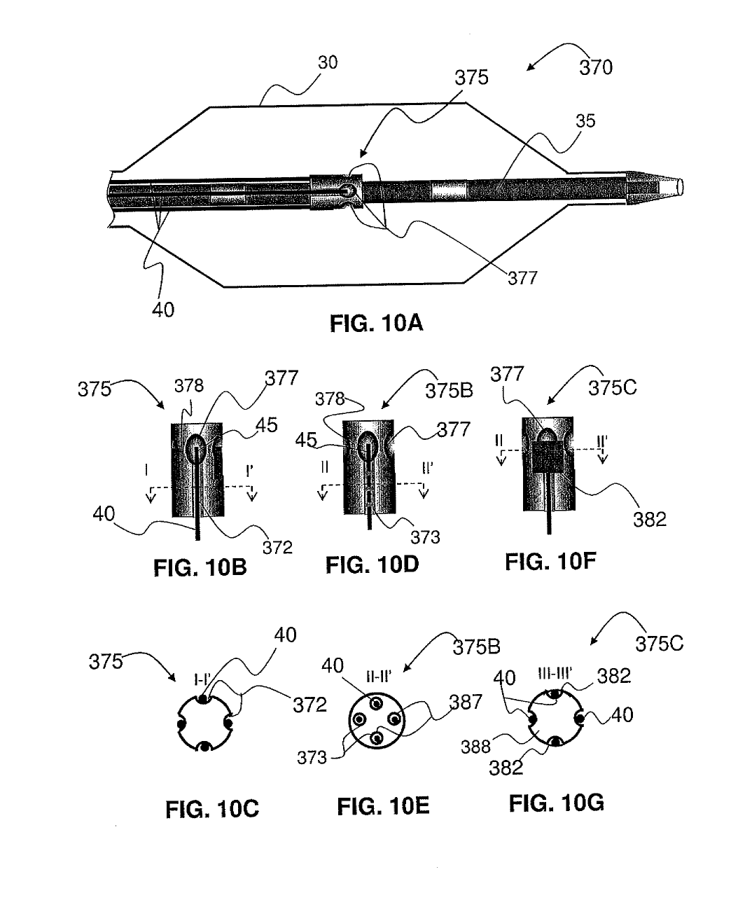

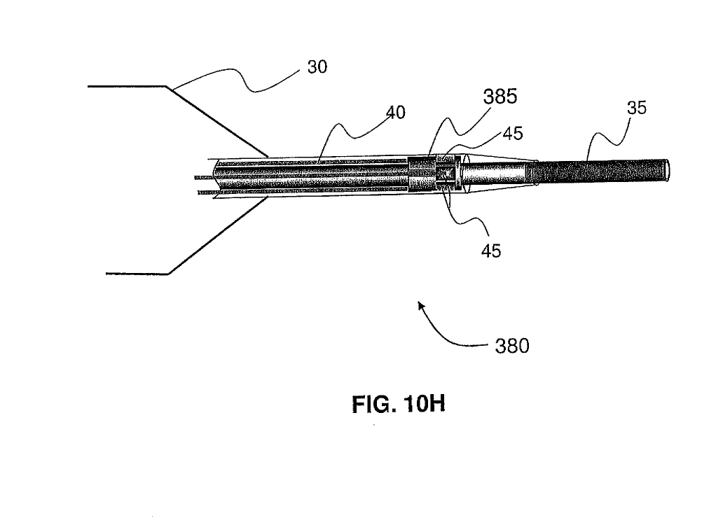

FIG. 10A is an expanded illustrative view of the treatment end of a catheter instrument 370, in accordance with embodiments of the present inventive concepts. FIG. 10B is an expanded illustrative view of a fiber alignment fixture, in accordance with embodiments of the present inventive concepts. FIG. 10C is a cross-sectional view of the fiber alignment fixture FIG. 10B, taken along section lines I-I′ of FIG. 10B, in accordance with embodiments of the present inventive concepts. A fiber alignment element 375 is formed about guidewire lumen 35 having recesses 377 in which fiber tips 45 can be positioned and aligned with recesses 377. In an embodiment, recesses 377 can have reflective surfaces that can help distribute or collect light to or from an area generally concentrated across an adjacent lumen (not shown). In an embodiment, the reflective surfaces are parabolic and shaped to more widely distribute signals toward an adjacent lumen. The shape of the parabola can be optimized based on the size and distribution/collection profile of fiber ends 45 and the estimated distance between distribution/collection ends 45 from each other and from the lumen wall (or the outside of outer balloon 30). Light blocking sections 378 prevent signals from traveling directly from a delivery fiber to a collection fiber without first being diffused about the area adjacent to a delivery fiber tip 45. Alignment grooves 372 align fibers 40 for centering fiber tips 45 within recess 377.

FIG. 10D is an expanded illustrative view of a fiber alignment fixture 385B, in accordance with embodiments of the present inventive concepts. FIG. 10E is a cross-sectional view of the fiber alignment fixture FIG. 10D, taken along section lines II-II′ of FIG. 10D, in accordance with embodiments of the present inventive concepts. Instead of grooves as described in the embodiment of FIGS. 10A-B, alignment holes 387 align fibers 40 for positioning into recess 377.

FIG. 10F is an expanded illustrative view of a fiber alignment fixture 385C, in accordance with embodiments of the present inventive concepts. FIG. 10G is a cross-sectional view of the fiber alignment fixture FIG. 10F, taken along section lines III-III′ of FIG. 10F, in accordance with embodiments of the present inventive concepts. In an embodiment, a partial covering 382 is placed over a portion of recess 377 so as to promote the scattering of signals prior to collection or delivery of signals through the recess 377 and the fiber tips 45. In an embodiment, only those recesses having delivery fibers are partially covered with partial covering 382. In an embodiment, the inner surface of partial covering 382 is reflective.

FIG. 10H is an expanded illustrative view of the distal end of a fiber probe arrangement 380, in accordance with embodiments of the present inventive concepts. The distal end of a catheter 380 has the ends of fibers 40 external to a balloon 30. The tips of fibers 40, for example, can be manufactured in a similar manner to other embodiments described herein. A fiber alignment fixture 385 can be manufactured according to other embodiments described herein such as, for example, in reference to the alignment fixtures of FIGS. 10A-G. The fibers 40 then will be arranged for measuring through blood media when the catheter 380 is placed within a lumen. Measurements of the lumen can take place prior to expansion of the balloon 30 so as to determine the pre-treatment characteristics (e.g., morphology, size, lesion type) prior to expansion of balloon 30 and also to determine the post-balloon expansion characteristics of the lumen. In an embodiment, a catheter is provided in this manner without a balloon so as to minimize the profile of the catheter.

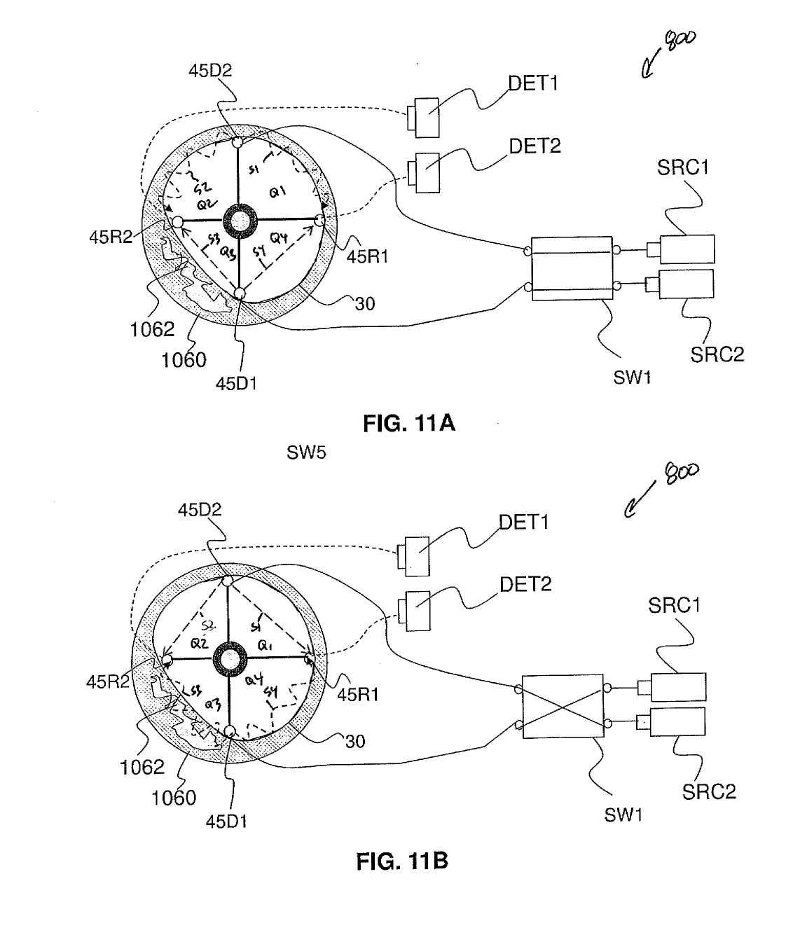

FIG. 11A is an illustrative schematic of an optical source and detector configuration of a catheter, in accordance with embodiments of the present inventive concepts. A catheter system 800 can comprise a catheter assembly 10 having a balloon 30, first and second radiation sources SRC1 and SRC2, first and second radiation detectors DET1 and DET2, and an optional radiation switch SW1. The catheter assembly 10 can further comprise a whisker body 80 having a plurality of whiskers 85, first and second delivery fibers 45D1 and 45D2, and first and second collector fibers 45R1 and 45R2.

The optical switch configuration as shown in FIG. 8A can direct radiation from at least one of the first and second radiation sources SRC1 and SRC2 to at least one of the first and second delivery fibers 45D1 and 45D2 so as to illuminate at least two adjacent circumferential quadrants Q1/Q2 and Q3/Q4 through which radiation is delivered to at least one of the first and second collection fibers 45R1 and 45R2 whereby at least one of the first and second detectors DET1 and DET2 detects said radiation. In an embodiment, the first and second detectors DET1 and DET2 can be components of an analyzer/processor system, such as, the analyzer/processor system 150 shown in FIG. 1B.

The catheter system 800 can comprise an optional switch SW1, which selects (swaps output) among one of two delivery fibers 45D1 and 45D2. For example, the switch SW1 can select the first radiation source SRC1 to deliver radiation through the first and second delivery fibers 45D1 and 45D2, the first delivery fiber 45D1 or the second delivery fiber. The switch SW1 can further select the second radiation source SRC2 to deliver radiation through the first and second delivery fibers 45D1 and 45D2, the first delivery fiber 45D1 or the second delivery fiber. The switch SW1 can further select the first radiation source SRC1 to deliver radiation through the first delivery fiber 45D1, and further select the second radiation source SRC2 to deliver radiation through the second delivery fiber 45D2.

The first delivery fiber 45D1, the first and second collector fibers 45R1 and 45R2, radiation signals/wavelengths emitted by the first and second radiation sources SRC1 and SRC2, and the first and second radiation detectors DET1 and DET2 can be selected to deliver and analyze radiation directed primarily through the balloon 30 media so as to measure relative area in at least one of the quadrants Q3 and Q4. The third and fourth radiation signals S3 and S4 are received by the second and first collector fibers 45R2 and 45R1, respectively, and are transmitted through the second and first delivery fibers 45R2 and 45R1 to corresponding radiation detectors DET1 and DET2. For example, third and fourth radiation signals S3 and S4 emitted from the first delivery fiber 45RD1 are partially absorbed by and reflected from portions of the wall of the balloon 30 and balloon media in the third and fourth quadrants Q3 and Q4, respectively. The amount of absorption of the signals can provide an estimate of the relative expansion of those areas (between the wall of balloon 30 and guidewire sheath 35 in Q3 and Q4. For example, a primary wavelength of about 1550 nanometers and a reference wavelength of about 1310 nanometers as described above can be used for such purpose.

The second delivery fiber 45D2, the first and second collector fibers 45R1 and 45R2, radiation wavelengths emitted by the first and second radiation sources SRC1 and SRC2, and the first and second radiation detectors DET1 and DET2 can be selected to deliver and analyze radiation directed through tissue adjacent to the wall of the balloon 30 so as to measure pathiophysiological properties of the tissue (e.g., collagen content, lipid content, calcium content, inflammatory factors, and the relative positioning of these features within the plaque) adjacent the quadrants Q1 and Q2. For example, first and second radiation signals S1 and S2 emitted from the second delivery fiber 45RD2 are partially absorbed by and reflected from portions of the lumen wall 1060 in the first and second quadrants Q1 and Q2, respectively. The first and second radiation signals S1 and S2 are received by the second and first collector fibers 45R2 and 45R1, respectively, and are transmitted through the second and first delivery fibers 45R2 and 45R1 to corresponding radiation detectors DET1 and DET2. For example, a scan of wavelengths between about 1550 nanometers and about 1850 nanometers can be used for such purpose.

FIG. 11B is an illustrative schematic of an optical source and detector configuration of FIG. 11A with sources SRC1 and SRC2 switched to deliver radiation signals to different delivery fibers according to an embodiment of the invention. After completion of a scan according to FIG. 11A, the first and second sources SRC1 and SRC2 can be switched to deliver radiation signals through fibers 45D1 and 45D2, respectively, so as to switch to scanning through the tissue adjacent Q3 and Q4 and to measure the relative distances between fibers and area across Q1 and Q2.

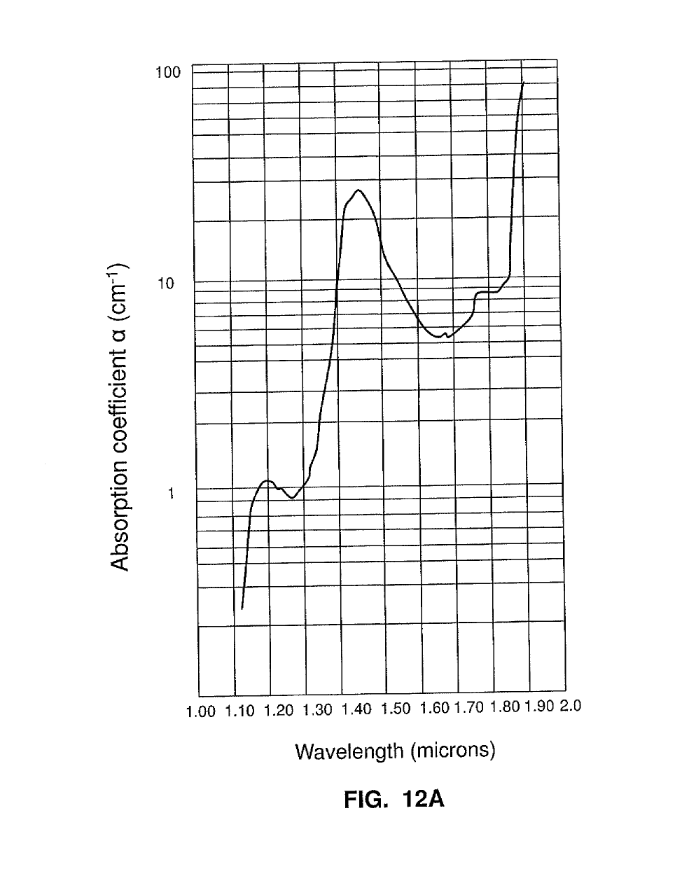

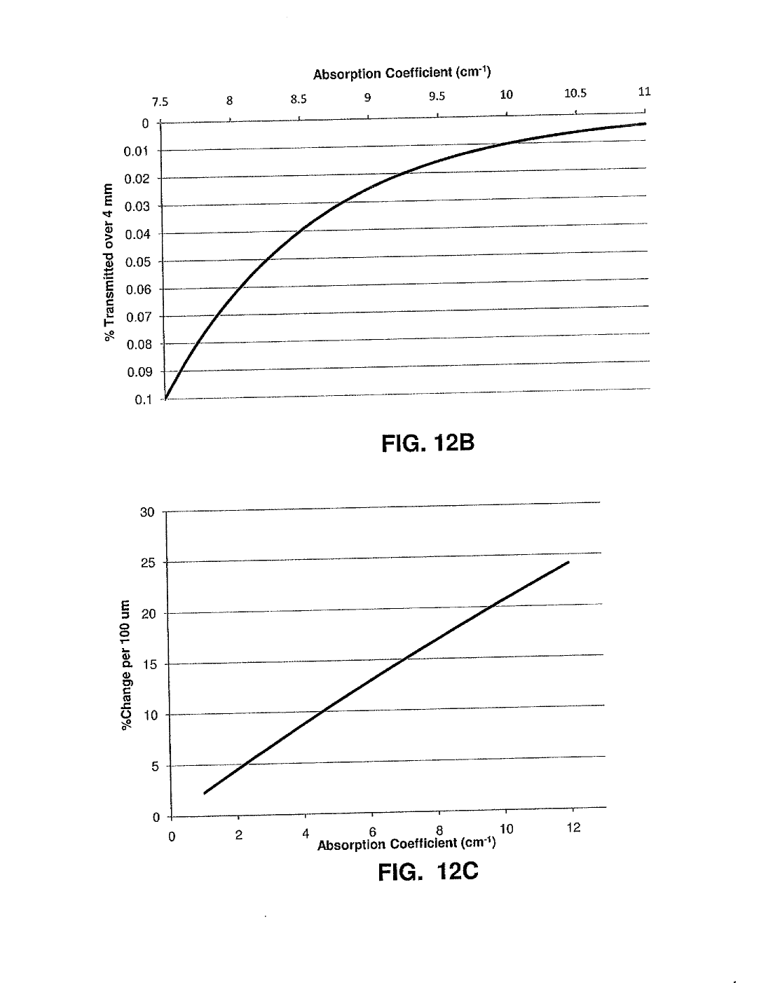

FIG. 12A is a logarithmic chart of measured absorption coefficients in water relative to selected wavelengths of light.

FIG. 12B is a chart comparing the absorption coefficient with the predicted % amount of signal delivered through 4 mm of water, in accordance with embodiments of the present inventive concepts. These calculations were made based on known absorption coefficients (see FIG. 12A) and the Beer-Lambert law for light traveling through an aqueous medium. FIG. 12C is a chart comparing the predicted change in intensity of light over each 100 mm of travel through water in comparison to the light's absorption coefficient, in accordance with embodiments of the present inventive concepts. These calculations were made based on known absorption coefficients (see FIG. 12A) and the Beer-Lambert law for light traveling through an aqueous medium.

In accordance with embodiments of the present inventive concepts for calculating the relative area of a region between a delivery fiber output and collection fiber input (e.g., between a delivery fiber 45D and collection fiber 45R of FIG. 7), optimal radiation signal wavelengths can be selected (based on the Beer Lambert law) that will demonstrate measurable changes in intensity (received by a detector) based on the change in distance between a delivery fiber output and collection fiber input that, for example, occurs in correspondence to the expansion of a balloon. For example, in an embodiment, a signal needs to travel as far as about 4 mm (e.g., across the inside of an expanded balloon of FIG. 8B) with a light source limited to about 10 mW (as a bio-safety restriction), a detector setup having an effective sensitivity to about 1 picowatt change in signal, and a measurable change in intensity across at least 100 mm increments between the output of the delivery fiber and input of the collection fiber. If a signal is delivered to achieve at least a 1 picowatt change, then light with an absorption coefficient in water of at least 5 cm⁻¹ is necessary (see FIG. 12C). In order to additionally detect a difference of about 1 picowatt over a 4 mm span, light with an absorption coefficient between about 5 and 10 cm⁻¹ can be used (see FIG. 12B). Observing the measured absorption coefficients of light shown in FIG. 12A, primary wavelengths in the near-IR spectrum of between about 1380 nanometers and about 1450 nanometers and between about 1550 nanometers and about 1850 nanometers are preferred for the described embodiment, including more specific wavelengths of about 1450 nanometers and/or about 1550 nanometers. A reference wavelength (the absorption of which does not change appreciably compared to the primary wavelength over the target distance) that is also detectable can be selected using the charts. For example, the absorption of a wavelength of 1310, with an absorption coefficient of about 1, will not change appreciably compared to a wavelength of 1550 over 4 mm. Thus, a reference wavelength can be selected to calculate a ratio between a primary and reference wavelength as described above and reduce the effect external influences on changes in the signal.

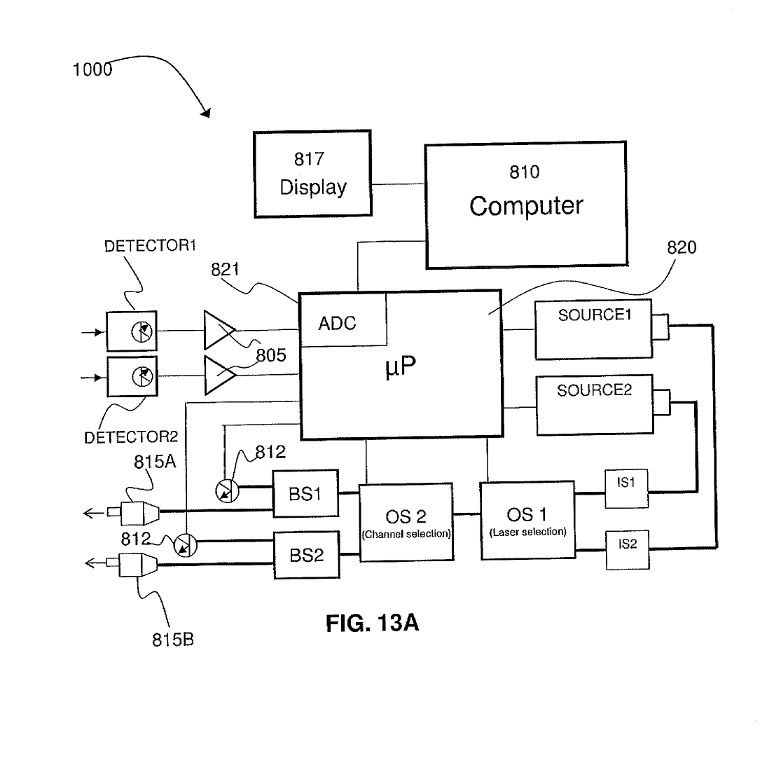

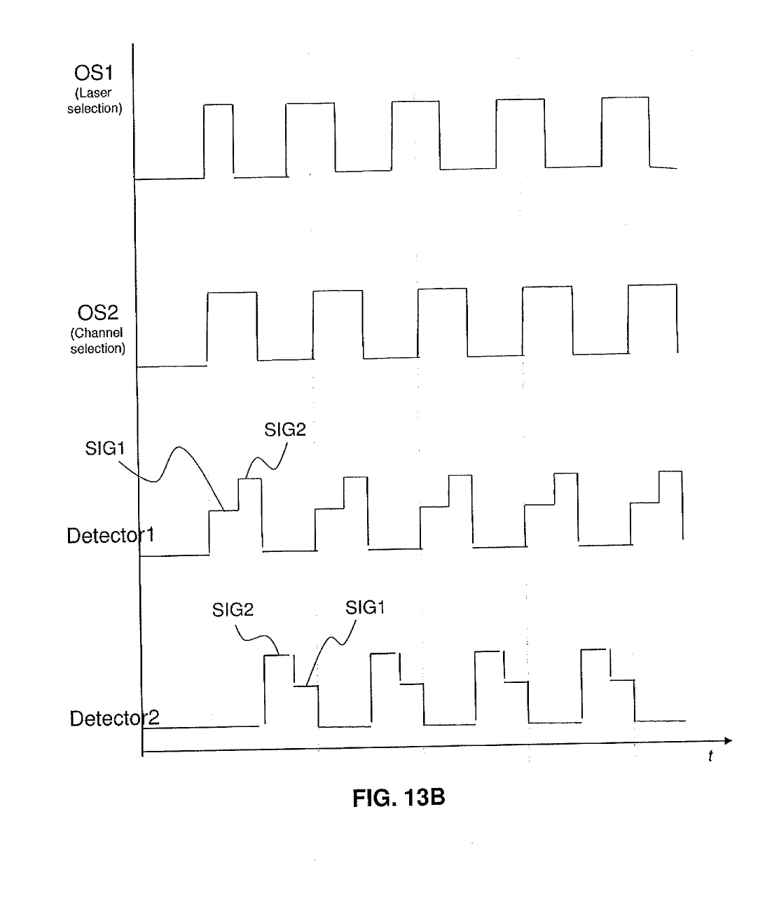

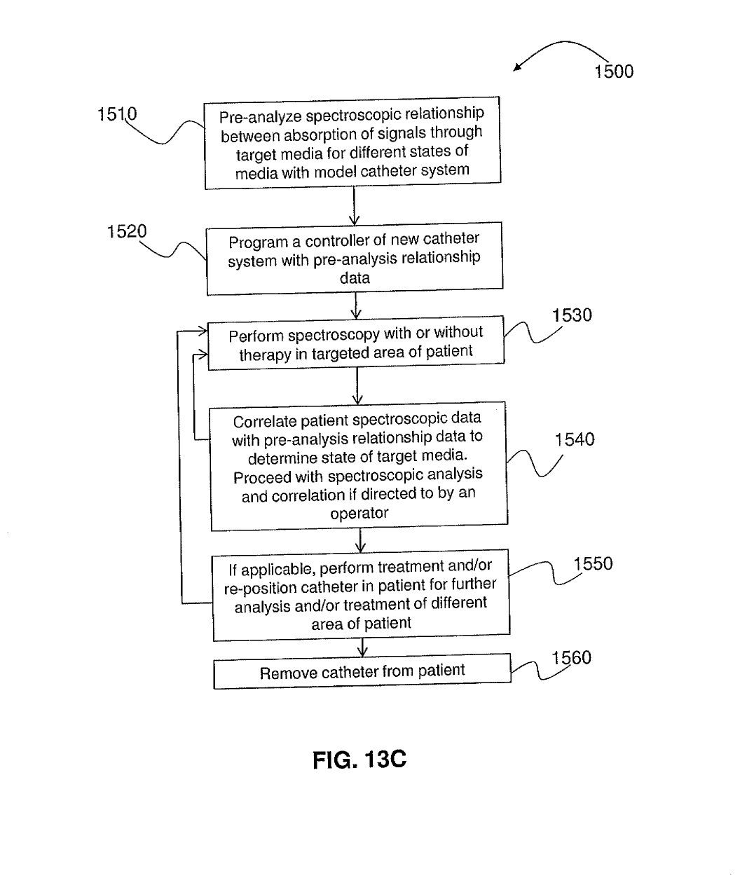

FIG. 13A is an illustrative schematic of a console configuration 1000, in accordance with embodiments of the present inventive concepts. The console 1000 includes signal sources SOURCE1 and SOURCE2 which, in an embodiment, are lasers. In an embodiment, SOURCE1 and SOURCE2 provide output signals of at least two wavelengths. In an embodiment, SOURCE1 and SOURCE2 provide output signals of between about 750 nanometers and about 2500 nanometers such as described above. Optionally, isolators IS1 or IS2 can be included to help isolate the signals created by sources SOURCE1 and SOURCE2 from noise.

Sources SOURCE1 and SOURCE2 are connected to an optical switch OS1 that directs one of the outputs from SOURCE1 and SOURCE2 to a second optical switch OS2. Optical switch OS2 directs output signals to one of two channels (e.g., delivery fibers) 815A and 815B. Optionally, a beam splitter (e.g., BS1 and BS2) can direct a portion of the output from switch OS2 to a controller/processor 820 in order to sample the output from the sources. In an embodiment, about 1% of the signal from switch OS2 is split from one or more beam splitters. In an embodiment, the signals from the beam splitters are directed to photo-diodes 812 for processing such as by controller/processor 820. The remaining signal is directed to output channels 815A or 815B. In another embodiment, a single optical switch (not shown) can replace OS1 and OS2 and have two inputs, one from each of SOURCE1 and SOURCE2, and two outputs, one to each of channels 815.

Detectors DETECTOR1 and DETECTOR2 are connected to amplifiers/buffers 805 (optinal), and amplifiers/buffers 805 are connected to an analog to digital controller (ADC) 821. The ADC 821 can be integrated into the controller/processor 820, or can be a separate device connected to the controller/processor 820.

In an embodiment, signals received (i.e., from collection fibers) through input channels of DETECTOR1 and DETECTOR2 are directed to controller/processor 820 for processing such as for calculating an absorbance using diffuse reflectance spectroscopy. In an embodiment, the controller/processor 820 can be connected to external processing and/or viewing devices such as a computer 810 with a display 817 (e.g., a monitor). The computer 810 and display 817 can, for example, function to take commands from operators, display results, further process data from the controller/processor 820, and/or control the console 1000 operations. The controller/processor 820 can be connected with various components such as sources SOURCE1, SOURCE2, and optical switches OS1 and OS2 so as to route commands to these devices.

In an embodiment, a signal is delivered from SOURCE1 to one of channels 815A and 815B and out to a delivery fiber tip such as fiber tip 45D 1 shown in FIG. 11A during which time a collection fiber such as fiber 45R2 and an input channel DETECTOR1 is monitored for signals delivered by SOURCE1. Once a signal is delivered by one channel (fiber) and collected, signal delivery can be switched to the other of channels 815A and 815B and collected by fiber tip 45R1 and an input channel DETECTOR2. Once signals are delivered by and collected from SOURCE1, signals can then be delivered by SOURCE 2 and collected by S. In an embodiment, one of the collected signals is used as a base reference such as, for example, the signal received in association with a delivered wavelength of about 1060 nanometers. During processing, a ratio between the base reference signal and at least one other signal associated with a different wavelength is calculated.

FIG. 13B is a chart of signals delivered and detected over a period of cycles through the system of FIG. 13A according to an embodiment of the invention. In an embodiment and initial configuration, switch OS1 is first signaled “on” to deliver radiation from SOURCE1 to switch OS2. Switch OS2 is signaled “on” to deliver the radiation from switch OS1 to output channel 815A. The signal from output channel 815A is carried to delivery fiber tip 45D1 (shown in FIG. 11A), the output from which is received by collection fiber tip 45R1 and delivered to DETECTOR1. The signal SIG1, shown as the detected signal of lower amplitude, received by DETECTOR1 is then processed by Analog-to-Digital Converter 821 and controller/processor 820. After a brief period (e.g., about 200 milliseconds) of delivering, receiving, and processing signals from SOURCE1, switch OS1 is signaled “off” to deliver radiation from SOURCE2 to switch OS2, the output from which continues to be directed to delivery fiber 45D1. During this period, collection fiber 45R1 receives signal SIG2, shown as the detected signal of greater amplitude, which is then processed by DETECTOR1 and controller/processor 820.

Once signals from SOURCE2 are delivered to delivery fiber 45D1 and collected by fiber 45R1 for a brief period of time, switch OS2 is switched “off” so that signals from SOURCE2 are delivered to delivery fiber 45D2 and collected by collection fiber 45R2. After a brief period of delivery and collection, switch OS1 is turned on again so that signals from SOURCE1 are delivered to delivery fiber 45D2 and collected by collection fiber 45R2. After another period of delivery and collection, switch OS2 is switched “on” again so that both switches OS1 and OS2 are in their original configuration for another cycle of delivery and collection. In an embodiment, these cycles can be repeated continuously while the balloon is expanded and monitored until the system predicts that full expansion is achieved. In an embodiment, one of the signals (e.g., SIG1) can be of a primary wavelength as referred to above and the other signal (e.g., SIG2) can be of a reference wavelength.

FIG. 13C is a flow chart 1500 of pre-programming and operation of a catheter system, in accordance with embodiments of the present inventive concepts. In an embodiment, a relationship between measurements taken through the blood, tissue, and/or balloon inflation media (e.g., the level of presence of blood and/or volume of inflation media and level of expansion of the balloon within the inflation media is present) for a particular fiber probe configuration can be pre-analyzed (the process correlating to step 1510 of FIG. 13C). For example, repeated spectroscopic absorbance measurements can be taken by a model catheter system positioned within in a model lumen (e.g., an animal or human cadaver, and/or artificially manufactured lumen). The state of the model lumen can be measured using an independent technique (e.g., a mechanical, optical, biopsy, and/or radiometric device for measuring the dimensions or other properties of the lumen) and the absorption measurements (e.g., the ratio between the primary wavelength absorption and reference wavelength absorption as discussed above) correlating with the different states of the model lumen can be pre-programmed into a system controller (the process correlating with step 1520 of FIG. 13C). A new catheter system with the programmed correlation data can then be deployed in a patient and positioned for spectroscopic analysis. Spectroscopic analysis (step 1530 of FIG. 13C) can then be performed and collected data can be compared and correlated with the pre-programmed relationship data (step 1540 of FIG. 13C). Further spectroscopic measurements and correlation can be performed until the desired amount of information is collected. In an embodiment, a therapeutic treatment (e.g., angioplasty) can be performed concurrently with the spectroscopic analysis (e.g., for monitoring the level of expansion of a balloon while the balloon is being expanded). In an embodiment, calculations made based on the spectroscopy performed in step 1540 may be determinative of performing additional therapy such as angioplasty or stent insertion. In an embodiment, the catheter may be repositioned for further analysis and/or treatment (step 1550 of FIG. 13C) based on calculations made in step 1540. Once all analysis and/or treatment is performed, the catheter can be removed from the patient (step 1560 of FIG. 13C).

FIG. 14A is an illustrative view of the distal end of a catheter instrument 600 for manipulating slidable fibers 40M with flexible whiskers 615, in accordance with embodiments of the present inventive concepts. FIG. 14B is an illustrative view of the catheter instrument of FIG. 14A shown with flexible whiskers 615 deployed, in accordance with embodiments of the present inventive concepts. FIG. 14C is an illustrative view of a catheter instrument of FIGS. 14A-14B with whiskers 615 retracted prior to catheter extraction, in accordance with embodiments of the present inventive concepts. A whisker body 610 is slidable along guidewire sheath 35 and is movably coupled to flexible whiskers 615 which hold and prop open slidable fibers 40M against the inside surface of the wall of the balloon 30.

Prior to deployment, the whiskers 615 are positioned in a retracted mode within a distal portion 620B of the catheter instrument 600 such as in correspondence with FIG. 2B above. The whiskers 615 and the fibers 40M can be positioned in this manner prior to deployment so as to avoid damaging the fibers 40M when, for example, a stent (not shown) is crimped over the balloon 30.

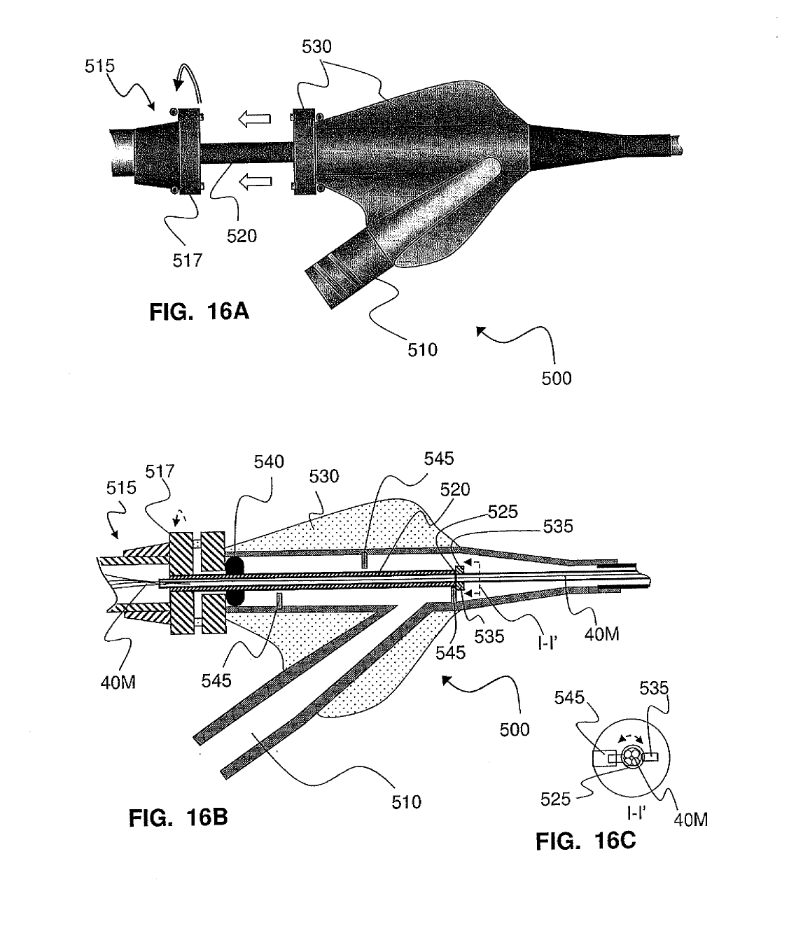

In an embodiment, the whisker body 610 and the whiskers 615 can be moved longitudinally by employing a means for pulling the fibers 40M (e.g., such as described below in reference to FIGS. 16A-16B), which in turn pull the attached whiskers 615 and slidable whisker body 610 along the guidewire sheath 35. The whisker body 610 and the whiskers 615 can be positioned within the balloon 30 and along an open longitudinal expanse 630 between distal 620B and proximal 620A portions of the catheter body so that the whiskers 615 are free to extend outwardly and position the tips of fibers 40M toward the inner surface of balloon 30 as shown in FIG. 14B and in correspondence with FIGS. 2A-2D above. After radiation analysis through the fibers 40M is complete, the fibers 40M can be pulled so as to pull and retract the whiskers 615 within the proximal portion 620A of the catheter body as shown in FIG. 14C, permitting the catheter instrument 600 to be removed without interference from the whiskers 615.

In an embodiment, the tips of whiskers 615 are fixed (e.g., with a suitable epoxy) to fibers 40M near the tips of fibers 40M so that when whiskers 615 extend outward toward the inner surface of the balloon 30, the tips of fibers 40M are held against the inner surface of the balloon 30 and also allow the whisker body 610 to be slidably moved along the guidewire sheath 35. FIG. 14C shows whiskers 615 and fibers 40M completely retracted within catheter sheath 620A In an embodiment, the whiskers 615 and the fibers 40M can be positioned in this manner prior to removal so as to avoid damaging the surrounding lumen or aspects of the catheter instrument 600.

FIG. 14D is an illustrative view of a catheter instrument 650 for manipulating slidable fibers 40M with flexible whiskers 615, in accordance with embodiments of the present inventive concepts. In this embodiment, a whisker base 610 is movably connected to a slidable sheath 625, which can extend to the proximate end 620A of the catheter instrument 650. The slidable sheath 625 is sufficiently stiff so as to permit both backward (proximately directed) and forward (distally directed) coupled movement of the whiskers 615, as shown by arrows 612. In an embodiment, the slidable sheath 625 can be integrated with the embodiments as shown in reference to FIGS. 16A-16B for providing a mechanism to movably manipulate the sheath 625. In an embodiment, the slidable sheath 625 is made from a thin flexible plastic material and can be further coated on the inside surface with a non-toxic lubricant.

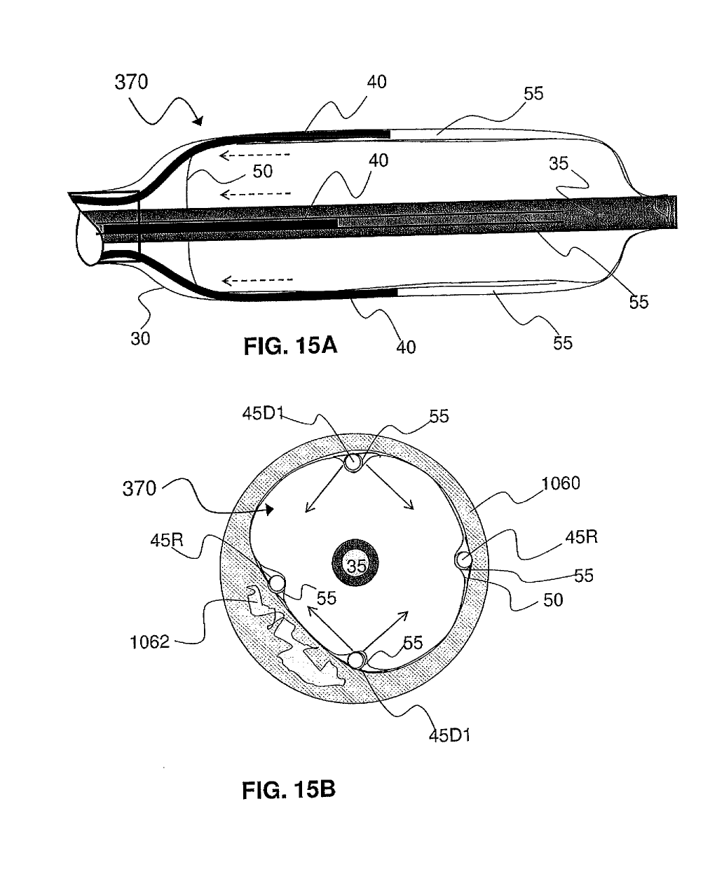

FIG. 15A is an illustrative view of the distal end of a catheter instrument with slidable fibers, in accordance with embodiments of the present inventive concepts. FIG. 15B is a cross-sectional illustrative view of the catheter instrument of FIG. 15A, in accordance with embodiments of the present inventive concepts. In an embodiment, a dual balloon arrangement such as, for example, described above in reference to FIGS. 8A-8B, includes translucent tubing 55 within which fibers 40 can slide and be re-positioned for taking measurements at different longitudinal positions within balloon 30.

FIG. 16A is an illustrative view of the proximate end of a catheter instrument 500 for manipulating slidable fibers, in accordance with embodiments of the present inventive concepts. The catheter instrument 500 comprises a slidably movable section 515 (shown in an open position). In an embodiment, the slidably movable section 515 is included for repositioning fibers 40M such as within the catheter components described in connection with FIGS. 14A-D.

FIG. 16B is a cross-sectional illustrative view of the catheter instrument of FIG. 16A, in accordance with embodiments of the present inventive concepts. FIG. 16C is a cross-sectional illustrative view of the catheter instrument of FIGS. 16A and 16B, taken along section lines I-I′ of FIG. 16B, in accordance with embodiments of the present inventive concepts. Section 515 includes an elongate tubular piece 520 that is fixedly connected to fibers 40M such as with an adhesive and/or a clamp 525. The remaining components of the catheter 500 remain stationary while a slidable handle section 515 may be pulled/pushed to draw fibers 40M toward the proximate end of the catheter instrument 500. The elongate tubular piece 520 remains within segment 530 and a gasket 540 prevents fluid (e.g., balloon expansion media) from exiting through the interface between segments 530 and 515. In an embodiment, catches 535 (attached to tubular piece 520) and 545 (attached to segment 515) can prevent segment 515 (including tubular piece 520) from sliding. In an embodiment, a handle 517 can rotate handle segment 515 and tubular piece 520 so as to disengage catches 535 and 545 and allow handle segment 515 to slide. In an embodiment, catches 545 are distributed along segment 530 so that when segment 515 is disengaged from a catch 545 and segment 515 proceeds to slide, another catch 545 positioned further toward the proximate end of the catheter will engage a catch 535 and stop the progress of sliding motion until handle 517 is rotated again. In an embodiment, catches 545 are also distributed so that the catch points correspond to predetermined longitudinal positions of fibers 40M along a balloon component. Pressure from fluid media entering through a port 510 may also apply pressure on segment 515 so that segment 515 slides proximately when catches 535 and 545 are not engaged.

It will be understood by those with knowledge in related fields that uses of alternate or varied materials and modifications to the systems and methods disclosed herein are apparent. This disclosure is intended to cover these and other variations, uses, or other departures from the specific embodiments as come within the art to which the present inventive concepts pertain.

What is claimed is:

1. A system for analyzing a body lumen comprising:
a flexible conduit that is elongated along a longitudinal axis, the flexible conduit having a proximal end and a distal end;
at least one delivery waveguide and at least one collection waveguide extending along the flexible conduit, a transmission output of the at least one delivery waveguide and a transmission input of the at least one collection waveguide located along a distal portion of the conduit;
a spectrometer connected to the at least one delivery waveguide and the at least one collection waveguide, the spectrometer configured to perform diffuse reflectance spectroscopy, wherein the spectrometer emits at least one primary radiation signal of a wavelength having an absorption coefficient of between about 8 cm⁻¹ and about 10 cm⁻¹ when transmitted through a highly aqueous media; and
a controller system configured to calculate at least one of an extent, area, and volume of highly aqueous media based on the amount of absorption of the at least one primary radiation signal measured through the highly aqueous media by the spectrometer.
